(19) United States
(12) Patent Application Publication
(10) Pub. No.: US 2025/0019341 A1
(43) Pub. Date: Jan. 16, 2025

(54) COMPOUNDS AND METHOD OF USE THEREOF FOR TREATING BACTERIAL DISEASES

(71) Applicants: PEKING UNIVERSITY, Beijing (CN); INSTITUTE OF GENETICS AND DEVELOPENTAL BIOLOGY, CHINESE ACADEMY OF SCIENCES, Beijing (CN)

(72) Inventors: Xiaoguang LEI, Beijing (CN); Jian-Min Zhou, Beijing (CN); Haijun Wang, Beijing (CN); Pei Miao, Beijing (CN); Wei Wang, Beijing (CN)

(21) Appl. No.: 18/736,209

(22) Filed: Jun. 6, 2024

Related U.S. Application Data
(63) Continuation of application No. PCT/CN2022/136852, filed on Dec. 6, 2022.

(30) Foreign Application Priority Data
Dec. 8, 2021 (WO) PCT/CN2021/136289

(51) Int. Cl. C07C 233/09; A01N 33/04; A01N 37/18; A01N 37/20; A01N 37/28; A01N 43/08; A01P 1/00; A61K 31/131; A61K 31/16; A61K 31/164; A61K 31/165; A61K 31/17; A61K 31/365; C07C 211/21; C07C 231/02; C07C 233/05; C07C 233/11; C07C 233/18; C07C 233/20; C07C 239/14; C07C 273/18; C07C 275/20; C07D 307/33

(52) U.S. Cl. CPC C07C 233/09; A01N 33/04; A01N 37/18; A01N 37/20; A01N 37/28; A01N 43/08; A01P 1/00; A61K 31/131; A61K 31/16; A61K 31/164; A61K 31/165; A61K 31/17; A61K 31/365; C07C 211/21; C07C 231/02; C07C 233/05; C07C 233/11; C07C 233/18; C07C 233/20; C07C 239/14; C07C 273/1854; C07C 275/20; C07D 307/33

(57) ABSTRACT

The present disclosure provides compounds that can inhibit the type III secretion system (TTSS) to decrease the pathogenesis of gram-negative bacteria. These compounds may have wide applications for treating bacteria diseases caused by gram-negative bacteria in a host species, including but not limited to, plants and animals. The present invention further relates to compositions that inhibit pathogenesis of gram-negative bacteria without killing the bacteria. Methods relating to preventing and/or treating infection of a host species by bacterial pathogens are also provided herein.

### CROSS-REFERENCE TO RELATED APPLICATIONS

This application is a Continuation of International Patent Application No. PCT/CN2022/136852 filed Dec. 6, 2022, which claims priority to and benefit of International Patent Application No. PCT/CN2021/136289, filed Dec. 8, 2021, the disclosures of which are hereby incorporated herein by reference in their entries.

### INTRODUCTION

Type three secretion system (TTSS) is a protein appendage found in several gram-negative bacteria. In pathogenic bacteria, the needle-like structure is used as a sensory probe to detect the presence of eukaryotic organisms and secrete proteins that help the bacteria infect them. The secreted effector proteins are secreted directly from the bacterial cell into the eukaryotic (ihost) cell, where they exert a number of effects that help the pathogen to survive and to escape an immune response. There remains a need to develop compounds that inhibit the type III secretion system and/or treat bacterial diseases.

### SUMMARY

The present disclosure provides compounds that can inhibit the type III secretion system (TTSS) to decrease the pathogenesis of gram-negative bacteria. These compounds may have wide applications for treating bacteria diseases caused by gram-negative bacteria in a host species, including but not limited to, plants and animals.

In one aspect, there is provided a compound, or a stereoisomer or tautomer thereof, or a salt of any of the foregoing, wherein the compound is selected from the group consisting of:

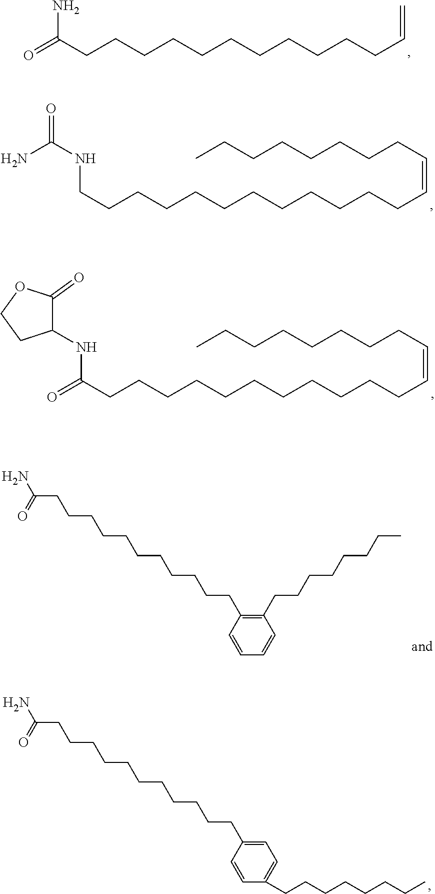 [SMILES: C=CCCCCCCCCCCCC(N)=O ; CCCCCCCC/C=C\CCCCCCCCCCCCNC(N)=O ; CCCCCCCC/C=C\CCCCCCCCCCCC(=O)NC1CCOC1=O ; CCCCCCCCc1ccccc1CCCCCCCCCCCC(N)=O ; CCCCCCCCc1ccc(CCCCCCCCCCCC(N)=O)cc1]

or a stereoisomer or tautomer thereof, or a salt of any of the foregoing.

Also provided herein is a compound of formula (IA), or a stereoisomer or tautomer thereof, or a salt of any of the foregoing,

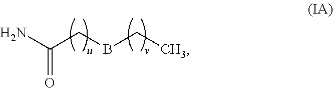

wherein B is phenylene; u and v are each independently an integer of at least 1.

Also provided herein is a composition comprising a compound of formula (IA) as described herein, or formula (I), or a stereoisomer or tautomer thereof, or a salt of any of the foregoing, wherein the composition inhibits pathogenesis of gram-negative bacteria without killing the bacteria,

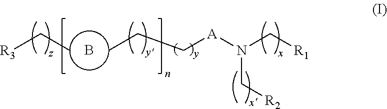

x is an integer selected from 0-8; x′ is an integer selected from 0-8; each of R₁ and R₂ is independently —Rₐ, —ORₐ, —N(Rₐ)₂, —NHC(O)Rₐ, —NHS(O)₂Rₐ, —NHS(O)₂N(Rₐ)₂, —C(O)ORₐ, —OC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, nitro, cyano, or halogen, wherein each Rₐ is independently hydrogen, C₁₋₇ alkyl, C₃₋₈ cycloalkyl, 3-12 membered heterocyclyl, C₆₋₁₂ aryl, or 5-12 membered heteroaryl, amino; wherein the 3-12 membered heterocyclyl of Rₐ is optionally substituted with one or more oxo; R₃ is independently —Rₐ, —ORₐ, —N(Rₐ)₂, —NHC(O)Rₐ, —NHS(O)₂Rₐ, —NHS(O)₂N(Rₐ)₂, —C(O)ORₐ, —OC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, nitro, cyano, or halogen, wherein each Rₐ is independently hydrogen, —CH═CH₂, —C≡CH, C₁₋₇ alkyl, C₃₋₈ cycloalkyl, 3-12 membered heterocyclyl, C₆₋₁₂ aryl, or 5-12 membered heteroaryl, amino; A is a chemical bond, —C(O)—, —C(S)—, or —NHC(O)—; y, y′, and z are each independently integer selected from 0-15; B is independently —CH═CH—, —C≡C—, C₁₋₄ alkyl, C₃₋₈ cycloalkyl, 3-12 membered heterocyclyl, C₆₋₁₂ aryl, or 5-12 membered heteroaryl, each of which is independently optionally substituted by one or more R_{b}, wherein each R_{b} is independently C₁₋₆ alkyl, C₁₋₆ alkoxyl, C₃₋₈ cycloalkyl, 3-12 membered heterocyclyl, C₆₋₁₂ aryl, 5-12 membered heteroaryl, amino, hydroxyl, carboxyl, nitro, cyano, or halogen; and n is an integer selected from 0-8.

In some embodiments, the composition described herein comprises a compound selected from the group consisting of:

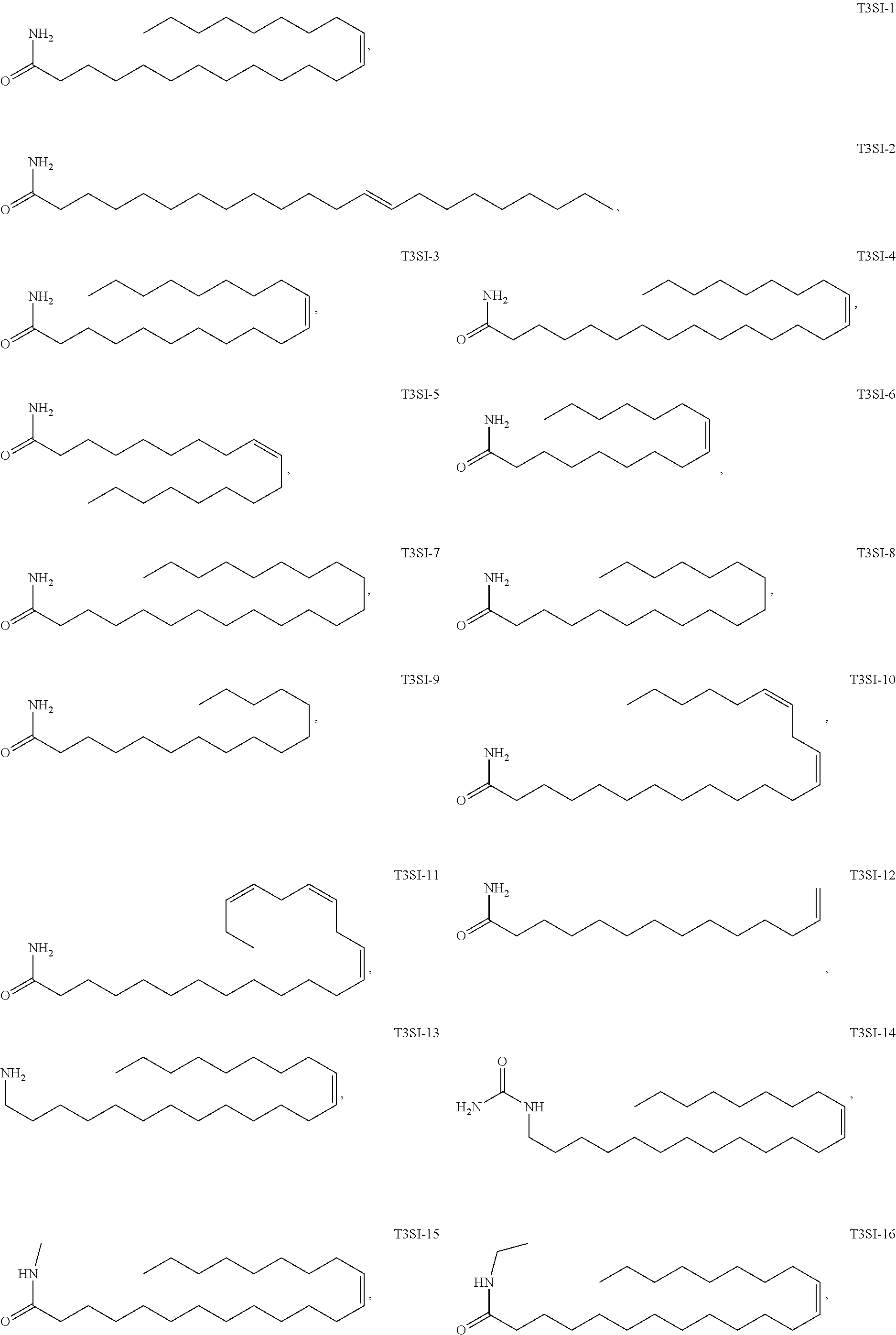

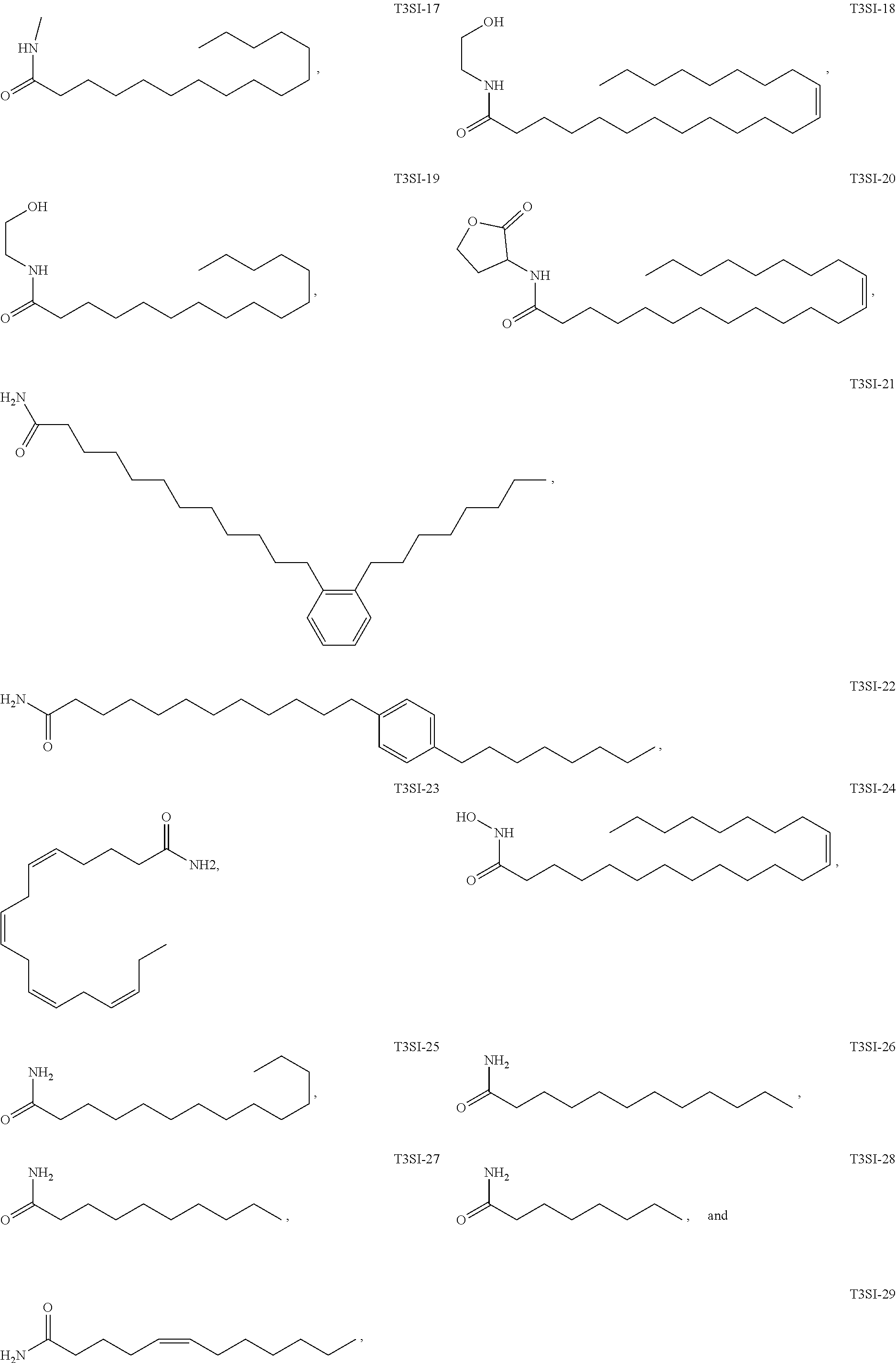

or a stereoisomer or tautomer thereof, or a salt of any of the foregoing.

In some embodiments, the composition described herein comprises at least about 20% (w/w) of the compound. In some embodiments, the composition comprises no more than about 10% (w/w) of the compound. In some embodiments, the composition comprises no more than about 60 μmol of the compound. In some embodiments, the composition is substantially free of bacteriocides. In some embodiments, the composition inhibits secretion of Type III secretion system (TTSS) of the gram-negative bacteria. In some embodiments, the composition is substantially free of a chemically synthesized ingredient. In some embodiments, the composition comprises a compound that is erucamide.

Also provided herein is a method for preventing and/or treating infection of a host species by bacterial pathogens, wherein the method comprises administering an effective amount of a composition as described herein to the host species. In some embodiments, the host species is a plant species. In some embodiments, the plant is selected from the group consisting of a solanaceous plant, a leguminous plant, a cruciferous plant, a gramineous plant, a cucurbitaceous plant, a liliaceous plant, and a rutaceous plant, a poaceae plant, an araliaceae plant. In some embodiments, the plant is a solanaceous plant, ad wherein the solanaceous plant(s) is tomato, eggplant, potato, tobacco, bell pepper, or chili pepper. In some embodiments, the plant is selected from the group consisting of a fruit tree, a horticultural tree and an ornamental plant. In some embodiments, the composition is administered to the plant by foliar administration, spraying, stem-coating, clipping, emersion, or watering. In some embodiments, the composition is administered by coating the seeds of the plant with the composition prior to sowing the seeds. In some embodiments, the composition is administered to the plant by adding the composition to the medium in which the plant is growing. In some embodiments, the host species is an animal species. In some embodiments, the animal species is a domesticated or agricultural animal. In some embodiments, the animal species is an insect species of agricultural importance, for example the honey bee. In some embodiments, the animal species is an aquatic animal. In some embodiments, the animal species is a poultry animal. In some embodiments, the composition is administered orally. In some embodiments, the composition is administered by infusion, injection, or inhalation. In some embodiments, Gram-negative bacterium is of the genus Pseudomonas, Xanthomonas, Ralstonia, Salmonella, Shigella, Escherichia, Burkholderia, Yersinia, Erwinia, Dickeya or Chlamydia.

Also provided herein is a method of preparing a compound of formula (IA), or formula (I), or a compound selected from the group consisting of: T3SI-1, T3SI-2, T3SI-3, T3SI-4, T3SI-5, T3SI-6, T3SI-7, T3SI-8, T3SI-9, T3SI-10, T3SI-11, T3SI-12, T3SI-13, T3SI-14, T3SI-15, T3SI-16, T3SI-17, T3SI-18, T3SI-19 T3SI-20, T3SI-21, T3SI-22, T3SI-23, and T3SI-24, or a stereoisomer or tautomer thereof, or a salt of any of the foregoing.

### BRIEF DESCRIPTION OF THE DRAWINGS

FIGS. 1A-1F shows that erucamide inhibits Type Three Secretion System (TTSS) secretion. (FIG. 1A) Extract of myb28/29 plants inhibits AvrPto secretion. Pst DC3000 bacteria were grown in the absence (mock) or presence indicated serial dilution, total protein (bacteria) and supernatant protein were subjected to immunoblot with indicated antibodies to analyze the abundance of AvrPto and RNAP. (FIG. 1B) Flow chart for TTSS secretion inhibitor extraction and purification. (FIG. 1C) The structure of erucamide. (FIG. 1D) Erucamide inhibits AvrPto secretion in vitro. Pst DC3000 bacteria were grown in the absence (mock) or presence of erucamide at indicated concentrations, total protein (bacteria) and supernatant protein were analyzed by immunoblots with the indicated antibodies. (FIG. 1E) Erucamide does not inhibit Pst DC3000 TTSS gene transcription. Bacteria carrying the indicated reporter genes were incubated with erucamide at the indicated concentrations, and luciferase activity was measured. Data are means±SD (n=3). (FIG. 1F) Erucamide does not affect Pst DC3000 growth. Growth curves for Pst DC3000 in KB medium with or without 150 μM erucamide.

FIGS. 2A-2C shows that erucamide blocks TTSS secretion in both plant and animal gram-negative pathogenic bacteria. (FIGS. 2A-2C) Erucamide inhibits Pst T1 effector AvrPto (FIG. 2A), Xcc8004 effector AvrAc (FIG. 2B) and S. typhimurium effector SopF (FIG. 2C) secretion in vitro. Bacteria were incubated with erucamide at indicated concentrations, total protein (bacteria) and supernatant protein were analyzed by immunoblots with the indicated antibodies.

FIGS. 3A-3F shows that erucamide inhibits P. syringae TTSS secretion and confers resistance to P. syringae in plant. (FIG. 3A) Concentration of erucamide in Arabidopsis leaves. Four-week-old leaves of the indicated genotypes were collected, extracted with dichloromethane and methanol (1:1), and the concentration of erucamide was determined by Gas chromatography-triple quadrupole mass spectrometry (GC-MS/MS). (FIG. 3B) Secretion of AvrPto in indicated genotypes. Four-week-old leaves of indicated genotypes were infiltration with bacterial strain expressing AvrPto-CyaA, and cAMP amounts were determined. (FIGS. 3C-3F) Pst DC3000 (FIG. 3C and FIG. 3E) and Pst DC3000 hrcC mutant (FIG. 3D and FIG. 3F) growth on indicated genotypes.

FIGS. 4A-4D shows inhibition of plant bacterial pathogen virulence by erucamide. (FIG. 4A) Erucamide inhibited Pst DC3000 hrcQ growth on N. benthamiana. The Pst DC3000 hrcQ mutant strain with or without 150 μM erucamide was inoculated into tobacco leaves and bacterial growth assay was performed. (FIG. 4B) Erucamide decreased the disease symptoms caused by P. actinidiae on kiwifruit leaves. P. actinidiae bacteria with (+) or without (−) 1 mM erucamide, were sprayed on leaves of kiwifruit, disease symptoms were photographed 10 days after inoculation. (FIG. 4C) Erucamide had protective effects on rice blight disease. Fully expanded rice leaves were clipped and dipped with Xoo P6 bacteria with (+) or without (−) 1 mM erucamide, disease symptoms were photographed 12 days after inoculation. (FIG. 4D) Erucamide decreased the virulence of R. solanacearum. Tomato plants were infected upon soil drenching by R. solanacearum with (+) or without (−) 1 mM erucamide, disease symptoms were photographed 6 days after inoculation.

FIGS. 5A-5E shows that erucamide blocked TTSS injectisome assembly. (FIG. 5A) Erucamide inhibited TTSS injectisome assembly. Pst DC3000 bacteria were grown with 0.2 mM erucamide (+) or solvent (−). Hrp pilus and flagellum of the bacterium was observed by transmission electron microscopy (Hrp pilus, red arrows, flagellum, blue arrows). Images are representative of three independent experiments. Scale bars, 0.2 m. (FIGS. 5B-5E) Erucamide treatment reduced accumulation of injectisome structural proteins in membranes. Pst DC3000 bacteria were grown with 0.2 mM erucamide or solvent, cellular and membrane proteins were extracted, relative levels of injectisome structural proteins were analyzed by quantitative proteomics (FIG. 5B and FIG. 5C) and the abundance of HrpA and HrcQbwas determined with indicated antibodies (FIG. 5D and FIG. 5E).

FIGS. 6A-6AD show inhibitory activity of erucamide analogs on P. syringe TTSS secretion. (FIGS. 6A-6AD) Pst DC3000 bacteria were grown in the presence of indicated concentration analogs or solvent, total protein (bacteria) and supernatant protein were extracted and analyzed by immunoblots with the indicated antibodies. T3SI-1, T3SI-2, T3SI-7, T3SI-8, T3SI-9, T3SI-12, T3SI-14, T3SI-16, T3SI-18, T3SI-19, T3SI-25 and T3SI-26 effectively inhibited AvrPto secretion in Pst DC3000 (FIGS. 6A, 6B, 6G, 6H, 6I, 6L, 6N, 6P, 6R, 6S, 6Y and 6Z), while T3SI-5, T3SI-6, T3SI-21, T3SI-22, T3SI-23, T3SI-24 and T3SI-29 treatment decrease the abundance of AvrPto in total protein (FIGS. 6E, 6F, 6U, 6V, 6W, 6X and 6AD), other analogs were not altered neither the efficient of AvrPto nor the accumulation of AvrPto in bacteria.

### DETAILED DESCRIPTION

The following description sets forth exemplary embodiments of the present disclosure. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

The term “about” refers to a variation of 11%, +3%, ±5%, or ±10% of the value specified. For example, “about 50” can in some embodiments includes a range of from 45 to 55. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. Reference to “about” a value or parameter herein includes (and describes) embodiments that are directed to that value or parameter per se. For example, description referring to “about X” includes description of “X”.

The singular forms “a” and “the” include plural references unless the context clearly dictates otherwise. Thus, e.g., reference to “the compound” includes a plurality of such compounds and includes reference to one or more compounds and equivalents thereof known to those skilled in the art.

“Alkyl” refers to an unbranched or branched saturated hydrocarbon chain. As used herein, alkyl has 1 to 10 carbon atoms (i.e., C₁₋₁₀ alkyl or C₁-C₁₀ alkyl), 1 to 8 carbon atoms (i.e., C₁₋₈ alkyl or C₁-C₈ alkyl), 1 to 6 carbon atoms (i.e., C₁₋₆ alkyl or C₁-C₆ alkyl), or 1 to 4 carbon atoms (i.e., C₁₋₄ alkyl or C₁-C₄ alkyl). Examples of alkyl groups include, without limitation, methyl, ethyl, propyl, isopropyl, n-butyl, sec-butyl, isobutyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂). It is understood that the term “alkyl” also contemplates a divalent moiety. “Alkoxyl” refers to the group “—O-alkyl”. Examples of alkoxyl groups include, without limitation, methoxy, ethoxy, n-propoxy, isopropoxy, n-butoxy, tert-butoxy, sec-butoxy, n-pentoxy, n-hexoxy and 1,2-dimethylbutoxy.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl or C₆-C₂₀ aryl), 6 to 12 carbon ring atoms (i.e., C₆₋₁₂ aryl or C₆-C₁₂ aryl), or 6 to 10 carbon ring atoms (i.e., C₆₋₁₀ aryl or C₆-C₁₀ aryl). Examples of aryl groups include, without limitation, phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl. It is understood that the term “aryl” also contemplates a divalent moiety.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl or C₃-C₂₀ cycloalkyl), 3 to 12 ring carbon atoms (i.e., C₃₋₁₂ cycloalkyl or C₃-C₁₂ cycloalkyl), 3 to 10 ring carbon atoms (i.e., C₃₋₁₀ cycloalkyl or C₃-C₁₀ cycloalkyl), 3 to 8 ring carbon atoms (i.e., C₃₋₈ cycloalkyl or C₃-C₈ cycloalkyl), or 3 to 6 ring carbon atoms (i.e., C₃₋₆ cycloalkyl or or C₃-C₆ cycloalkyl). Monocyclic groups include, without limitation, cyclopropyl, cyclobutyl, cyclopentyl, cyclohexyl, cycloheptyl and cyclooctyl. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom. It is understood that the term “cycloalkyl” also contemplates a divalent moiety.

“Heteroaryl” refers to an aromatic group having a single ring, multiple rings or multiple fused rings, with one or more ring heteroatoms independently selected from nitrogen, oxygen and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl) and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above. Examples of heteroaryl groups include, but are not limited to, pyridyl, pyrimidyl, thiophenyl, furanyl, thiazolyl, oxazolyl, isoxazolyl, thiophenyl, pyrrolyl, pyrazolyl, 1,3,4-oxadiazolyl, imidazolyl, isothiazolyl, triazolyl, 1,3,4-thiadiazolyl, tetrazolyl, benzofuranyl, benzothiophenyl, pyrazolopyridinyl, indazolyl, benzothiazolyl, benzooxazolyl, and benzoimidazolyl and the like. It is understood that the term “heteroaryl” also contemplates a divalent moiety.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro and may comprise one or more (e.g., 1 to 3) oxo (═O) or N-oxide (N⁺—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ or C₂-C₂₀ heterocyclyl), 2 to 12 ring carbon atoms (i.e., C₂₋₁₂ or C₂-C₁₂ heterocyclyl), 2 to 10 ring carbon atoms (i.e., C₂₋₁₀ or C₂-C₁₀ heterocyclyl), 2 to 8 ring carbon atoms (i.e., C₂₋₈ or C₂-C₈ heterocyclyl), 3 to 12 ring carbon atoms (i.e., C₃₋₁₂ or C₃-C₁₂ heterocyclyl), 3 to 8 ring carbon atoms (i.e., C₃₋₈ or C₃-C₈ heterocyclyl), or 3 to 6 ring carbon atoms (i.e., C₃-6 or C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. In certain instances, heterocyclyl includes 3-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. The term “heterocyclyl” also includes “spiroheterocyclyl” when there are two positions for substitution on the same carbon atom. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl and the like. It is understood that the term “heterocyclyl” also contemplates a divalent moiety.

“Oxo” refers to ═O.

“Halogen” or “halo” includes fluoro, chloro, bromo and iodo.

The terms “optional” or “optionally” means that the subsequently described event or circumstance may or may not occur.

“Substituted” as used herein means one or more (e.g., 1-8, 1-6, 1-5, 1-4, 1-3, 1-2, 2-5, 2-4, 2-3, 3-5, or 3-4) hydrogen atoms of the group is replaced with the substituents listed for that group, which may be the same or different. “Optionally substituted” means that a group may be unsubstituted or substituted by one or more (e.g., 1-8, 1-6, 1-5, 1-4, 1-3, 1-2, 2-5, 2-4, 2-3, 3-5, or 3-4) substituents listed for that group, wherein the substituents may be the same or different.

Provided are also are stereoisomers, mixture of stereoisomers, tautomers, hydrates, solvates, isotopically enriched analogs and salts of the compounds described herein.

The compounds disclosed herein, or their salts, may include an asymmetric center and may thus give rise to enantiomers, diastereomers and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-performance liquid chromatography (HPLC). When the compounds described herein contain olefinic double bonds or other centers of geometric asymmetry and unless specified otherwise, it is intended that the compounds include both E- and Z-geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposable mirror images of one another and “diastereomers,” which refers to stereoisomers that have at least two asymmetric atoms, but which are not mirror-images of each other. Thus, all stereoisomers (for example, geometric isomers, optical isomers and the like) of the present compounds (including those of the salts, solvates and hydrates of the compounds), such as those which may exist due to asymmetric carbons on various substituents, including enantiomeric forms (which may exist even in the absence of asymmetric carbons), rotameric forms, atropisomers and diastereomeric forms, are contemplated.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds disclosed herein may be atropisomers and are considered as part of this disclosure. Stereoisomers can also be separated by use of chiral HPLC.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any compound or structure given herein, is also intended to represent unlabeled forms as well as isotopically labeled forms of the compounds. These forms of compounds may also be referred to as an “isotopically enriched analog.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Such compounds may exhibit increased resistance to metabolism and are thus useful for increasing the half-life of any compound when administered to a mammal, particularly a human. Such compounds are synthesized by means well known in the art, for example by employing starting materials in which one or more hydrogens have been replaced by deuterium.

The terms “inhibit,” “inhibiting,” and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

“Host species” as used herein includes animals and plants. A host species may be a vertebrate, invertebrate, plant, insect, mammal, or non-mammal.

As used herein, “treatment” or “treating” is an approach for obtaining beneficial or desired results. For purposes of this disclosure, beneficial or desired results include, but are not limited to, inhibition of virulence of bacteria and inhibition of secretion of Type III secretion system. Also encompassed by “treatment” is a reduction of pathological consequence of a bacterial disease.

The term “effective amount” used herein refers to an amount of a compound or composition sufficient to prevent and/or treat infection of a host species by bacterial pathogens. An effective amount can be administered in one or more administrations.

The term “carrier,” as used herein, refers to relatively nontoxic chemical compounds or agents that facilitate the incorporation of a compound into cells or tissues.

The term “excipient” as used herein means an inert or inactive substance that may be used in the production of a drug or pharmaceutical, such as a tablet containing a compound of the disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The term “bacterial disease” as used herein, refers to a pathological condition in a host species that is caused by bacteria. Bacteria causing bacterial diseases include, but are not limited to, Gram-negative bacteria.

### Compounds

In one aspect, provided is a compound, or a stereoisomer or tautomer thereof, or a salt of any of the foregoing, wherein the compound is selected from the group consisting of:

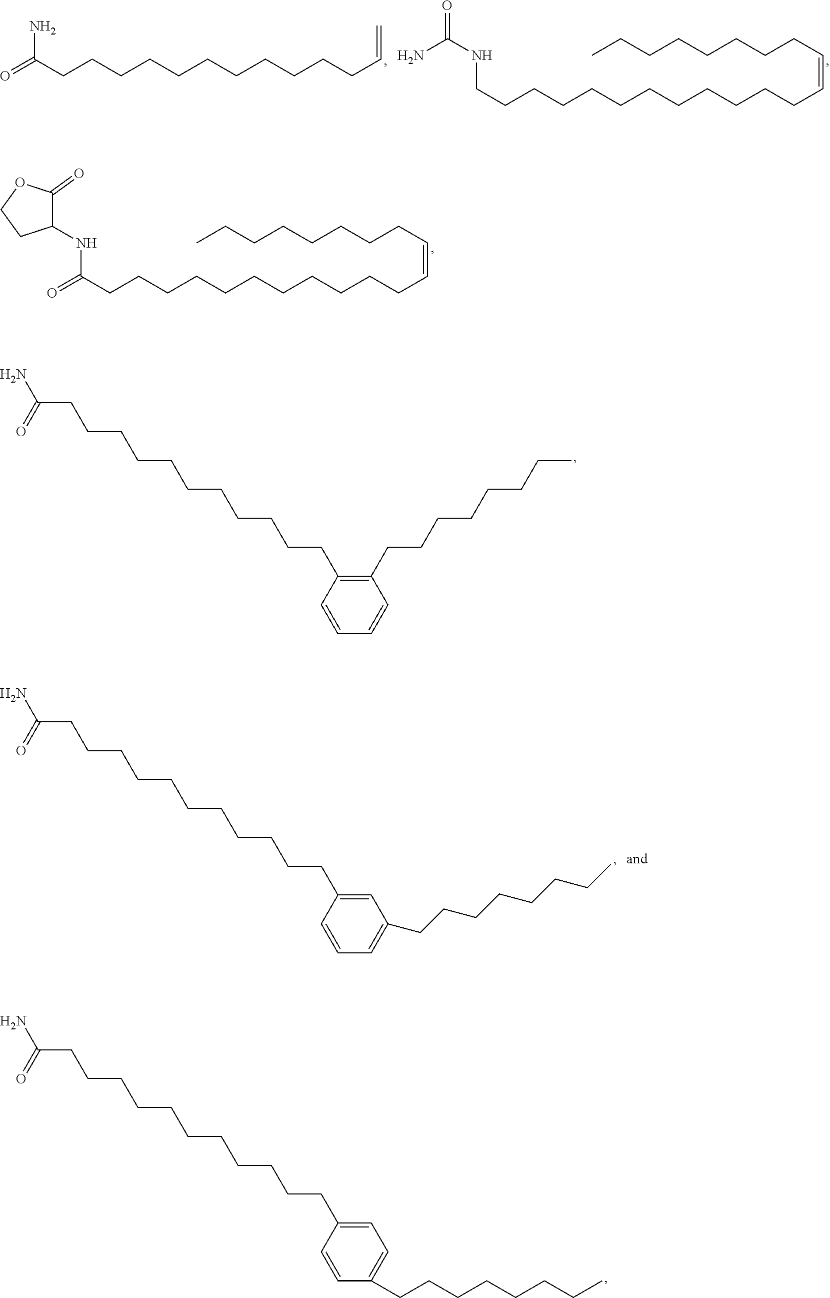

or a stereoisomer or tautomer thereof, or a salt of any of the foregoing.

In another aspect, provided is a compound of formula (IA), or a stereoisomer or tautomer thereof, or a salt of any of the foregoing,

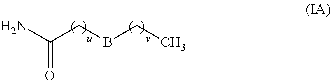

wherein:
B is phenylene or biphenyl, and u and v are each independently an integer of at least 1.

In some embodiments, the phenylene of the compound of formula (IA), or a stereoisomer or tautomer thereof, or a salt of any of the foregoing, is substituted at any suitable positions, e.g., ortho-substituted, para-substituted, and meta-substituted. In some embodiments, u is an integer of 1 to 40, 1 to 35, 1 to 30, 1 to 25, 1 to 20, 1 to 15, or 1 to 10. In some embodiments, v is an integer of 1 to 40, 1 to 35, 1 to 30, 1 to 25, 1 to 20, 1 to 15, or 1 to 10. In some embodiments, the compound of formula (IA), or a stereoisomer or tautomer thereof, or a salt of any of the foregoing, is

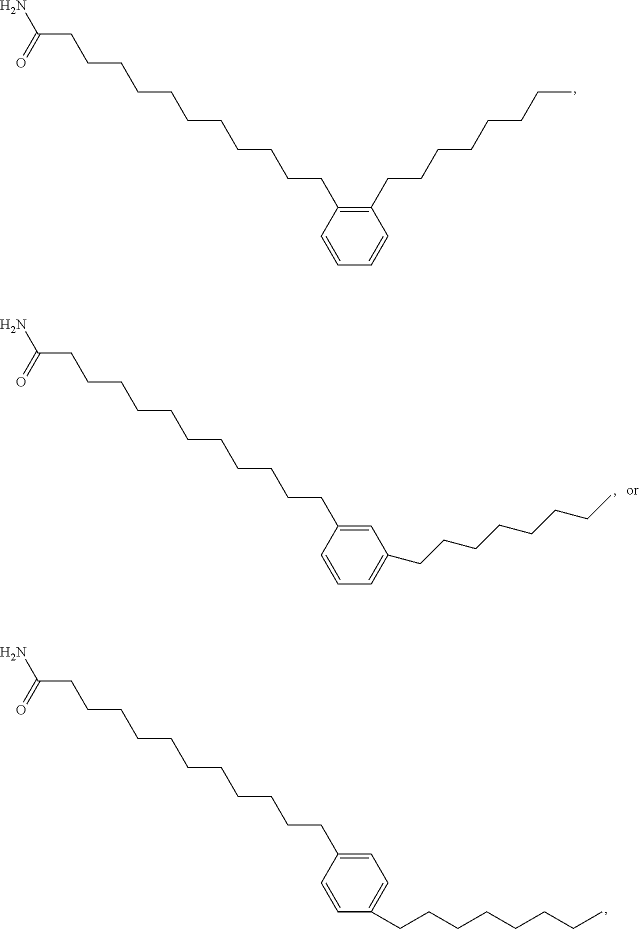 [SMILES: CCCCCCCCc1ccccc1CCCCCCCCCCCC(N)=O ; CCCCCCCCc1cccc(CCCCCCCCCCCC(N)=O)c1 ; CCCCCCCCc1ccc(CCCCCCCCCCCC(N)=O)cc1]

or a stereoisomer or tautomer thereof, or a salt of any of the foregoing.

In one aspect, provided is a compound of formula (I), or a stereoisomer or tautomer thereof, or a pharmaceutically acceptable salt of any of the foregoing,

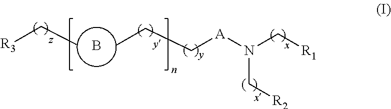

wherein:
x is an integer selected from 0-8;
x′ is an integer selected from 0-8;
each of R₁ and R₂ is independently —Rₐ, —ORₐ, —N(Rₐ)₂, —NHC(O)Rₐ, —NHS(O)₂Rₐ, —NHS(O)₂N(Rₐ)₂, —C(O)ORₐ, —OC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, nitro, cyano, or halogen, wherein each Rₐ is independently hydrogen, C₁₋₇ alkyl, C₃₋₈ cycloalkyl, 3-12 membered heterocyclyl, C₆₋₁₂ aryl, or 5-12 membered heteroaryl, amino; wherein the 3-12 membered heterocyclyl of Rₐ is optionally substituted with one or more oxo; R₃ is independently —Rₐ, —ORₐ, —N(Rₐ)₂, —NHC(O)Rₐ, —NHS(O)₂Rₐ, —NHS(O)₂N(Rₐ)₂, —C(O)ORₐ, —OC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, nitro, cyano, or halogen, wherein each Rₐ is independently hydrogen, —CH═CH₂, —C≡CH, C₁₋₇ alkyl, C₃₋₈ cycloalkyl, 3-12 membered heterocyclyl, C₆₋₁₂ aryl, or 5-12 membered heteroaryl, amino;
A is a chemical bond, —C(O)—, —C(S)—, or —NHC(O)—;
y, y′, and z are each independently integer selected from 0-15;
B is independently —CH═CH—, —C≡C—, C₁₋₄ alkyl, C₃₋₈ cycloalkyl, 3-12 membered heterocyclyl, C₆₋₁₂ aryl, or 5-12 membered heteroaryl, each of which is independently optionally substituted by one or more R_{b}, wherein each R_{b} is independently C₁₋₆ alkyl, C₁₋₆ alkoxyl, C₃₋₈ cycloalkyl, 3-12 membered heterocyclyl, C₆₋₁₂ aryl, 5-12 membered heteroaryl, amino, hydroxyl, carboxyl, nitro, cyano, or halogen;
n is an integer selected from 0-8.

In some embodiments of compound of formula (I) or any related formula, x is 0. In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 0-8. In some embodiments, x is 0-7. In some embodiments, x is 0-6. In some embodiments, x is 0-5. In some embodiments, x is 0-4. In some embodiments, x is 0-3. In some embodiments, x is 0-2. In some embodiments, x is 0-1. In some embodiments, x is 1-7. In some embodiments, x is 1-6. In some embodiments, x is 1-5. In some embodiments, x is 1-4. In some embodiments, x is 1-3. In some embodiments, x is 1-2. In some embodiments, x is 2-8. In some embodiments, x is 2-7. In some embodiments, x is 2-6. In some embodiments, x is 2-5. In some embodiments, x is 2-4. In some embodiments, x is 2-3. In some embodiments, x is 3-8. In some embodiments, x is 3-7. In some embodiments, x is 3-6. In some embodiments, x is 3-5. In some embodiments, x is 3-4. In some embodiments, x is 4-8. In some embodiments, x is 4-7. In some embodiments, x is 4-6. In some embodiments, x is 4-5. In some embodiments, x is 5-8. In some embodiments, x is 5-7. In some embodiments, x is 5-6.

In some embodiments of compound of formula (I) or any related formula, x′ is 0. In some embodiments, x′ is 1. In some embodiments, x′ is 2. In some embodiments, x′ is 3. In some embodiments, x′ is 4. In some embodiments, x′ is 5. In some embodiments, x′ is 6. In some embodiments, x′ is 7. In some embodiments, x′ is 8. In some embodiments, x′ is 0-8. In some embodiments, x′ is 0-7. In some embodiments, x′ is 0-6. In some embodiments, x′ is 0-5. In some embodiments, x′ is 0-4. In some embodiments, x′ is 0-3. In some embodiments, x′ is 0-2. In some embodiments, x′ is 0-1. In some embodiments, x′ is 1-7. In some embodiments, x′ is 1-6. In some embodiments, x′ is 1-5. In some embodiments, x′ is 1-4. In some embodiments, x′ is 1-3. In some embodiments, x′ is 1-2. In some embodiments, x′ is 2-8. In some embodiments, x′ is 2-7. In some embodiments, x′ is 2-6. In some embodiments, x′ is 2-5. In some embodiments, x′ is 2-4. In some embodiments, x′ is 2-3. In some embodiments, x′ is 3-8. In some embodiments, x′ is 3-7. In some embodiments, x′ is 3-6. In some embodiments, x′ is 3-5. In some embodiments, x′ is 3-4. In some embodiments, x′ is 4-8. In some embodiments, x′ is 4-7. In some embodiments, x′ is 4-6. In some embodiments, x′ is 4-5. In some embodiments, x′ is 5-8. In some embodiments, x′ is 5-7. In some embodiments, x′ is 5-6.

In some embodiments of compound of formula (I) or any related formula, R₁ is independently —Rₐ, —ORₐ. In some embodiments, R₁ is independently —Rₐ, —ORₐ, wherein each Rₐ is independently hydrogen. In some embodiments, each R₁ is independently hydrogen, methyl. In some embodiments, R₁ is hydrogen. In some embodiments, R₁ is C₁₋₆ alkyl. In some embodiments, R₁ is methyl. In some embodiments, R₁ is independently —Rₐ, —ORₐ, wherein each Rₐ is independently 3-12 membered heterocyclyl.

In some embodiments of compound of formula (I) or any related formula, R₂ is independently —Rₐ, —ORₐ. In some embodiments, R₂ is independently —Rₐ, —ORₐ, wherein each Rₐ is independently hydrogen. In some embodiments, each R₂ is independently hydrogen, methyl. In some embodiments, R₂ is hydrogen. In some embodiments, R₂ is C₁₋₆ alkyl. In some embodiments, R₂ is methyl. In some embodiments, R₂ is independently —Rₐ, —ORₐ, wherein each Rₐ is independently 3-12 membered heterocyclyl.

In some embodiments of compound of formula (I) or any related formula, A is —C(O)—.

In some embodiments of compound of formula (I) or any related formula, R₃ is hydrogen. In some embodiments, R₃ is methyl. In some embodiments, R₃ is —CH═CH₂.

In some embodiments of compound of formula (I) or any related formula, A is a chemical bond.

In some embodiments of compound of formula (I) or any related formula, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments, y is 11. In some embodiments, y is 12. In some embodiments, y is 13. In some embodiments, y is 14. In some embodiments, y is 15.

In some embodiments of compound of formula (I) or any related formula, y′ is 1. In some embodiments, y′ is 2. In some embodiments, y′ is 3. In some embodiments, y′ is 4. In some embodiments, y′ is 5. In some embodiments, y′ is 6. In some embodiments, y′ is 7. In some embodiments, y′ is 8. In some embodiments, y′ is 9. In some embodiments, y′ is 10. In some embodiments, y′ is 11. In some embodiments, y′ is 12. In some embodiments, y′ is 13. In some embodiments, y′ is 14. In some embodiments, y′ is 15.

In some embodiments of compound of formula (I) or any related formula, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments, z is 11. In some embodiments, z is 12. In some embodiments, z is 13. In some embodiments, z is 14. In some embodiments, z is 15.

In some embodiments of compound of formula (I) or any related formula, B is independently —CH═CH—. In some embodiments, B is independently C₁₋₄ alkyl. In some embodiments, B is independently C₆₋₁₂ aryl. In some embodiments, B is independently

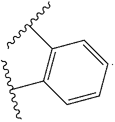 [SMILES: *c1ccccc1*]

In some embodiments, B is independently.

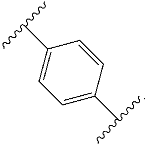 [SMILES: *c1ccc(*)cc1]

In some embodiments, each B is independently C₁₋₆ alkyl, 3-12 membered heterocyclyl, or 5-12 membered heteroaryl, each of which is independently optionally substituted by R_{b}. In some embodiments, each B is independently 3-12 membered heterocyclyl, which is optionally substituted by R_{b}. In some embodiments, each B is independently C₃₋₈ cycloalkyl, which is optionally substituted by R_{b}. In some embodiments, each B is independently C₆₋₁₂ aryl, which is optionally substituted by R_{b}. In some embodiments, each B is independently 5-12 membered heteroaryl, which is optionally substituted by R_{b}. In some embodiments, each B is independently —CH₂—, —CH₂CH₂—,

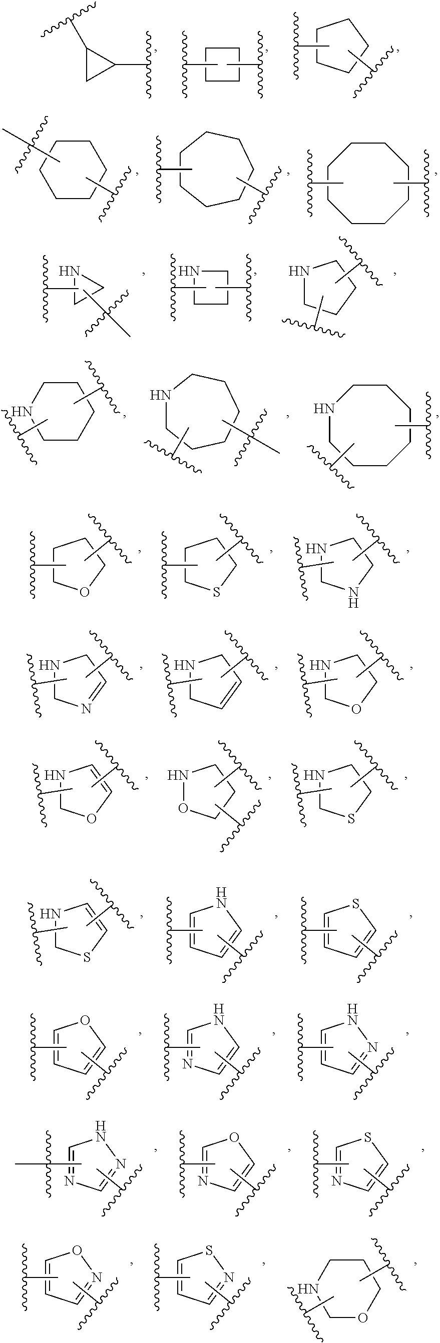

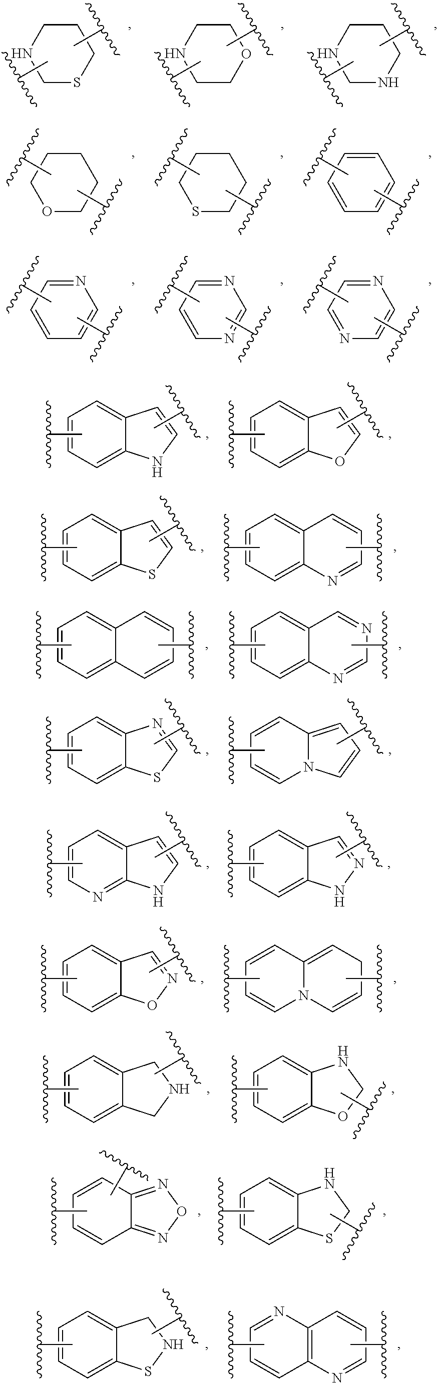

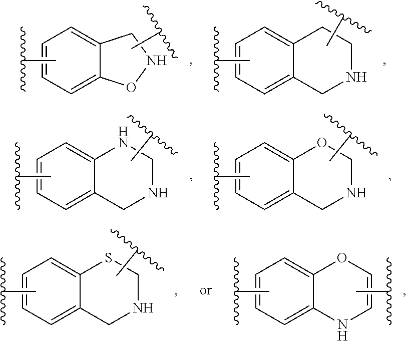

each of which is independently optionally substituted by R_{b}. It is understood that each wavy line indicates the point of attachment to the rest of the molecule and the point of attachment can be at any atom as valency permits. For example,

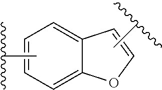

contemplates, without limitation,

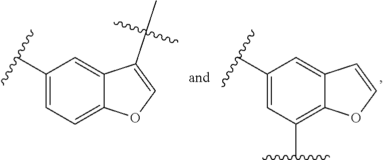 [SMILES: *c1ccc2occ(*)c2c1 ; *c1cc(*)c2occc2c1]

In some embodiments of compound of formula (I) or any related formula, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 0-8. In some embodiments, n is 0-7. In some embodiments, n is 0-6. In some embodiments, n is 0-5. In some embodiments, n is 0-4. In some embodiments, n is 0-3. In some embodiments, n is 0-2. In some embodiments, n is 0-1. In some embodiments, n is 1-7. In some embodiments, n is 1-6. In some embodiments, n is 1-5. In some embodiments, n is 1-4. In some embodiments, n is 1-3. In some embodiments, n is 1-2. In some embodiments, n is 2-8. In some embodiments, n is 2-7. In some embodiments, n is 2-6. In some embodiments, n is 2-5. In some embodiments, n is 2-4. In some embodiments, n is 2-3. In some embodiments, n is 3-8. In some embodiments, n is 3-7. In some embodiments, n is 3-6. In some embodiments, n is 3-5. In some embodiments, n is 3-4. In some embodiments, n is 4-8. In some embodiments, n is 4-7. In some embodiments, n is 4-6. In some embodiments, n is 4-5. In some embodiments, n is 5-8. In some embodiments, n is 5-7. In some embodiments, n is 5-6.

It is understood that specific values described herein are values for a compound of formula (I) or any related formula where applicable. Two or more values may combined. Thus, it is to be understood that any variable for a compound of formula (I) or any related formula may be combined with any other variable for a compound of formula (I) or any related formula the same as if each and every combination of variables were specifically and individually listed. As an example, in some embodiments, provided is a compound of formula (I), or a stereoisomer or tautomer thereof, or a salt of any of the foregoing, wherein x and x′ are each 0, 1, or 2; each of R₁ and R₂ is hydrogen or hydroxyl; y, y′, and z are each independently 2, 4, 6, 8, 10, 12; R₃ is hydrogen; A is a chemical bond or —C(O)—; B is —CH═CH—, C₁₋₄ alkyl, or C₆₋₁₂ aryl, each of which is independently optionally substituted by R_{b}; n is independently 1, 2, 4, 5, 6, 7, 8. In some embodiments, A is —C(O)— and B is —CH═CH—. In some embodiments, A is —C(O)—, B is —CH═CH—, and n is 1. In some embodiments, A is —C(O)—, B is —CH═CH—, and n is 2. In some embodiments, A is —C(O)—, B is —CH═CH—, and n is 3. In some embodiments, A is —C(O)—, B is —CH═CH—, and n is 4. In some embodiments, A is —C(O)—, and B is C₆₋₁₂ aryl. In some embodiments, A is —C(O)—, B is C₆₋₁₂ aryl, and n is 1. In some embodiments, A is —C(O)— and B is methyl.

Exemplary compounds provided by the present disclosure include, but are not limited to, a compound, shown in Table 1, or a stereoisomer, tautomer, hydrate, solvate, isotopically labeled form, or salt thereof. In some embodiments, provided is a compound shown in Table 1, or a stereoisomer or tautomer thereof, or a salt of any of the foregoing. In some embodiments, provided is a compound shown in Table 1, or a stereoisomer or tautomer thereof, or a salt of any of the foregoing.

TABLE 1

| | |
|---|---|
| 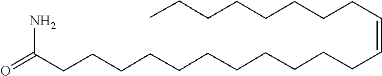 [SMILES: CCCCCCCC/C=C\CCCCCCCCCCCC(N)=O] | T3 SI-1 |
| 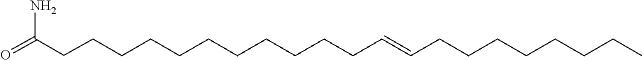 [SMILES: CCCCCCCC/C=C/CCCCCCCCCCCC(N)=O] | T3 SI-2 |
| 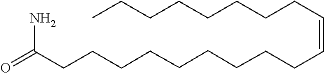 [SMILES: CCCCCCCC/C=C\CCCCCCCCCC(N)=O] | T3 SI-3 |
| 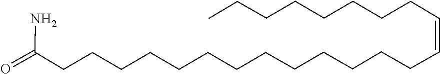 [SMILES: CCCCCCCC/C=C\CCCCCCCCCCCCCC(N)=O] | T3 SI-4 |
| 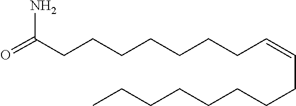 [SMILES: CCCCCCCC/C=C\CCCCCCCC(N)=O] | T3 SI-5 |
| 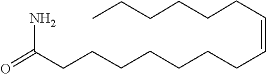 [SMILES: CCCCCC/C=C\CCCCCCCC(N)=O] | T3 SI-6 |
| 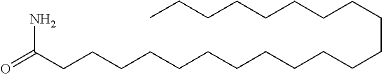 [SMILES: CCCCCCCCCCCCCCCCCCCCCC(N)=O] | T3 SI-7 |
| 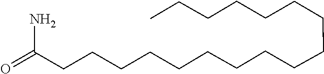 [SMILES: CCCCCCCCCCCCCCCCCC(N)=O] | T3 SI-8 |
| 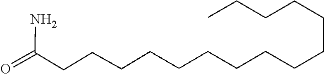 [SMILES: CCCCCCCCCCCCCCCC(N)=O] | T3 SI-9 |
| 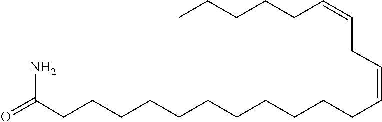 [SMILES: CCCCC/C=C\C/C=C\CCCCCCCCCCCC(N)=O] | T3SI-10 |
| 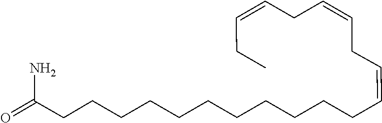 [SMILES: CC/C=C\C/C=C\C/C=C\CCCCCCCCCCCC(N)=O] | T3SI-11 |
| 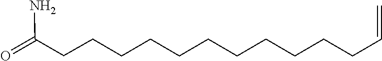 [SMILES: C=CCCCCCCCCCCCC(N)=O] | T3SI-12 |
| 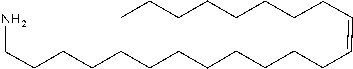 [SMILES: CCCCCCCC/C=C\CCCCCCCCCCCCN] | T3SI-13 |
| 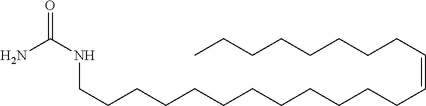 [SMILES: CCCCCCCC/C=C\CCCCCCCCCCCCNC(N)=O] | T3SI-14 |
| 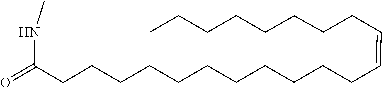 [SMILES: CCCCCCCC/C=C\CCCCCCCCCCCC(=O)NC] | T3SI-15 |
| 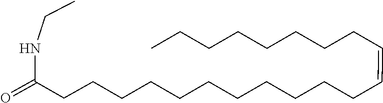 [SMILES: CCCCCCCC/C=C\CCCCCCCCCCCC(=O)NCC] | T3SI-16 |
| 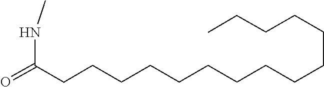 [SMILES: CCCCCCCCCCCCCCCC(=O)NC] | T3SI-17 |
| 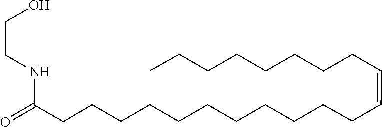 [SMILES: CCCCCCCC/C=C\CCCCCCCCCCCC(=O)NCCO] | T3SI-18 |
| 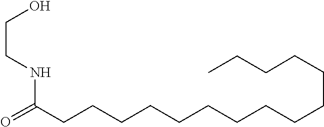 [SMILES: CCCCCCCCCCCCCCCC(=O)NCCO] | T3 SI-19 |
| 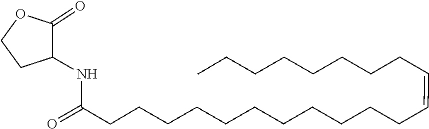 [SMILES: CCCCCCCC/C=C\CCCCCCCCCCCC(=O)NC1CCOC1=O] | T3 SI-20 |
| 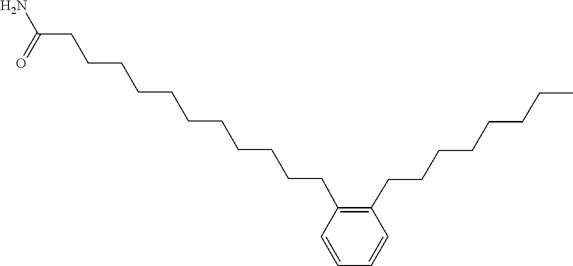 [SMILES: CCCCCCCCc1ccccc1CCCCCCCCCCCC(N)=O] | T3 SI-21 |
| 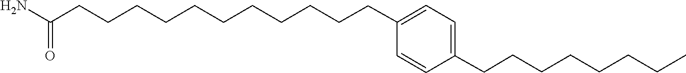 [SMILES: CCCCCCCCc1ccc(CCCCCCCCCCCC(N)=O)cc1] | T3 SI-22 |
| 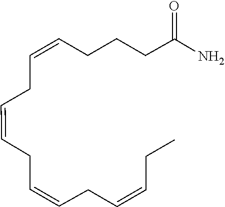 [SMILES: CC/C=C\C/C=C\C/C=C\C/C=C\CCCC(N)=O] | T3 SI-23 |
| 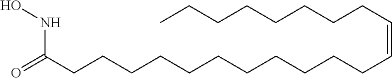 [SMILES: CCCCCCCC/C=C\CCCCCCCCCCCC(=O)NO] | T3 SI-24 |
| 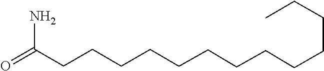 [SMILES: CCCCCCCCCCCCCC(N)=O] | T3 SI-25 |
| 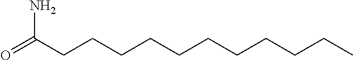 | T3 SI-26 |
| 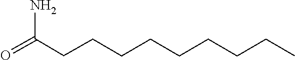 | T3 SI-27 |
| 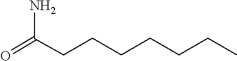 | T3 SI-28 |
| 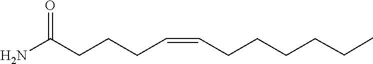 | T3 SI-29 |

### Compositions

In another aspect, provided is a composition, comprising a compound of formula (IA) or formula (I) as described herein, or a stereoisomer or tautomer thereof, or a salt of any of the foregoing, wherein the composition inhibits virulence of gram-negative bacteria without killing the bacteria.

In some embodiments, the composition provided herewith inhibits secretion of Type III secretion system (TTSS) of the gram-negative bacteria. In some embodiments, the composition inhibits P. syringae TTSS secretion. In some embodiments, the composition comprises a compound that is a broad-spectrum TTSS secretion inhibitor. In some embodiments, the composition inhibits virulence of gram-negative pathogenic bacteria (e.g., Pst DC3000, Pst T1, S. typhimurium, R. solanacearum, P. syringae pv. actinidiae, and X oryzae pv. oryzae). In some embodiments, the composition inhibits virulence of Pst DC3000, R. solanacearum, P. syringae pv. actinidiae, or X oryzae pv. oryzae. In some embodiments, the composition inhibits TTSS injectisome assembly.

In some embodiments, the composition is substantially free of bacteriocides, which include but are not limited to, disinfectants, antiseptics, or antibiotics. In some embodiments, the composition is substantially free of a chemically synthesized ingredient. Unless otherwise stated, “substantially free” refers to a composition which contains no more than 35%, 30%, 25%, 20%, 15%, 10%, 5%, 1%, 0.5%, or 0.1% of an undesired substance (e.g., bacteriocide or chemically synthesized ingredient).

In some embodiments, the compound is a compound described in formula (I), formula (IA), Table 1, or a stereoisomer or tautomer thereof, or a salt of any of the foregoing. In some embodiments, the composition provided herewith comprises a compound of Table 1, or a stereoisomer or tautomer thereof, or a salt of any of the foregoing. In some embodiments, the compound is erucamide. In some embodiments, the compound is an erucamide derivative. In some embodiments, the composition comprises at least about 20% w/w (including for example at least any of 255, 30%, 35%, 40%, 45%, or 50% w/w) of the compound of formula (I) or formula (IA). In some embodiments, the composition comprises no more than about 10% w/w (e.g., no more than about any of 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, and 10% w/w) of the compound of formula (I) or formula (IA). In some embodiments, the composition comprises no more than about 60 μmol (e.g., 10 μmol, 15 μmol, 20 μmol, 25 μmol, 30 μmol, 35 μmol, 40 μmol, 45 μmol, 50 μmol, 55 μmol, and 60 μmol) of the compound of formula (I) or formula (IA).

The composition provided herein may take any form, including but is not limited to, liquid, aerosol, gel, cream, or solid (e.g., powder, pellets, and crystals). The composition described herein may be in any form suitable for administration to the host species (e.g. plants or animals).

Also provided herein is a kit, comprising a compound disclosed herein, or a stereoisomer or tautomer thereof, or a salt of any of the foregoing, or a composition disclosed herein. In some embodiments, the kit comprises a unit dose of a compound or composition described herein and/or instructions for administering the same.

### Methods of Treatment

In another aspect, provided is a method of preventing and/or treating infection of a host species by bacterial pathogens, comprising administrating to the host species an effective amount of a composition described herein. In some embodiments, the treatment is effective to inhibit TTSS secretion of gram-negative pathogenic bacteria. A method of preventing and/or treating infection of a host species by bacterial pathogens may, in some embodiments, be a method of treating bacterial disease.

### Host Species

In some embodiments, the host species is a plant species, including but not limited to, vegetables, pulses, grains, tropical species (e.g., bananas), sub-tropical species (e.g., citrus fruits), other trees and shrubs, and flowering plants of horticultural interest. In some embodiments, the plant species is a solanaceous plant (e.g., tomato, eggplant, potato, tobacco, bell pepper, and chili pepper). In some embodiments, the plant species is a leguminous plant (e.g., beans, soybenas, peas, chickpeas, peanuts, and lentils). In some embodiments, the plant species is a cruciferous plant (e.g., bok choy, broccoli, brussels sprouts, cabbage, and cauliflower). In some embodiments, the plant species is a gramineous plant (e.g., cereals, bamboo, and sugar cane). In some embodiments, the plant species is a cucurbitaceous plant (e.g., squash, zucchini, pumpkin, gourd, watermelon, cantaloupe, and cucumber). In some embodiments, the plant species is a liliaceous plant (e.g., tulip, aloe, and asparagus). In some embodiments, the plant species is a rutaceous plant (e.g., citrus). In some embodiments, the plant species is a poaceae plant (e.g., rice, wheat, and maize). In some embodiments, the plant species is an araliaceae plant (e.g., Ginseng and Panax Notoginseng). In some embodiments, the plant species is a fruit tree, a horticultural tree, or an ornamental plant.

In some embodiments, the host species is an animal species, including but not limited to, a domesticated animal, an agricultural animal, an insect species of agricultural importance, an aquatic animal (e.g., fish, turtle, shrimp, clam, and lobster), and a poultry animal (e.g., chickens, ducks, turkeys, and geese). In some embodiments, the animal species is an insect species of agricultural importance, such as various species of bees, including but not limited to honey bees. In some embodiments, the animal species is a mammal (e.g., cows, sheep, pigs, and goats). In some embodiments, the animal species is a non-mammal (e.g., birds, fish, and reptiles). In some embodiments, the animal species is a farm animal (ex., cattle, poultry, or swine). In some embodiments, the host species is a vertebrate. In some embodiments, the host species is an invertebrate. In some embodiments, the host species is a non-human animal.

### Routes of Administration

The composition described herein may be administered via any routes suitable to the host species. For example, when the host species is a plant species, the composition may be administered to the plant species by foliar administration, spraying, stem-coating, clipping, emersion, or watering. In some embodiments, the composition is administered exogenously (e.g., spraying or brushing). When the host species is an animal species, the composition may be administered to the animal species by infusion, injection, or inhalation. In some embodiments, the composition is administered, e.g., by intravenous injection, intramuscular injection, infusion, or subcutaneous injection. In some embodiments, the composition is administered orally. In some embodiments, the composition is administered topically.

The composition described herein may be administered to any and/or all portions of the host species. For example, the composition may be administered to any and/or all portions of a plant, including root system, shoots, stems, nodes, internodes, petiole, leaves, flowers, and fruits, either prior to or post-harvest. The composition may also be administered to plant seeds. In some embodiments, the composition is administered to the plant by adding the composition to the medium in which the plant is growing. In some embodiments, the composition is administered to the plant by coating the seeds of the plant with the composition prior to sowing the seeds.

### Bacterial Diseases

In some embodiments, the treatment is effective for a plant bacterial disease. In some embodiments, the plant bacterial disease is caused by P. actinidiae. In some embodiments, the plant bacterial disease is rice bacteria blight disease caused by Xanthomonas oryzae pv. oryzae (Xoo). In some embodiments, the plant bacterial disease is caused by Pst DC3000, Pst T1, S. typhimurium, R. solanacearum, P. syringae pv. actinidiae, or X oryzae pv. oryzae. In some embodiments, the treatment is effective for an animal bacterial disease. In some embodiments, the treatment is effective for an animal bacterial disease caused by S. typhimurium. In some embodiments, the bacterial disease is caused by Pseudomonas syringae, Xanthomonas campestris, Ralstonia solanacearum or Salmonella Typhimurium. In some embodiments, the bacterial disease is caused by a Gram-negative bacteria (e.g., Pseudomonas, Xanthomonas, Ralstonia, Salmonella, Shigella, Escherichia, Burkholderia, Yersinia, Erwinia, Dickeya, and Chlamydia).

In some embodiments, the gram-negative bacteria is Pseudomonas syringae, Pseudomonas aeruginosa, Pseudomonas amygdali, Pseudomonas avellanae, Pseudomonas caricapapayae, Pseudomonas cichorii, Pseudomonas coronafaciens, Pseudomonas ficuserectae, Pseudomonas helianthi, Pseudomonas meliae, Pseudomonas savastanoi, Pseudomonas tomato, Pseudomonas viridiflava, Pseudomonas asplenii, Pseudomonas cannabina, Pseudomonas costantinii, Pseudomonas fuscovaginae, Pseudomonas suis, Pseudomonas marginalis, Pseudomonas mediterranea, Xanthomonas albilineans, Xanthomonas alfalfae, Xanthomonas ampelina, Xanthomonas arboricola, Xanthomonas axonopodis, Xanthomonas boreopolis, Xanthomonas badrii, Xanthomonas bromi, Xanthomonas campestris, Xanthomonas cassavae, Xanthomonas citri, Xanthomonas codiaei, Xanthomonas cucurbitae, Xanthomonas cyanopsidis, Xanthomonas cynarae, Xanthomonas euvesicatoria, Xanthomonas fragariae, Xanthomonas gardneri, Xanthomonas holcicola, Xanthomonas hortorum, Xanthomonas hyacinthi, Xanthomonas maliensis, Xanthomonas malvacearum, Xanthomonas maltophila, Xanthomonas manihotis, Xanthomonas melonis, Xanthomonas oryzae, Xanthomonas papavericola, Xanthomonas perforans, Xanthomonas phaseoli, Xanthomonas pisi, Xanthomonas populi, Xanthomonas sacchari, Xanthomonas theicola, Xanthomonas translucens, Xanthomonas vasicola, Xanthomonas vesicatoria, Ralstonia solanacearum, Salmonella enterica, Shigella dysenteriae, Enterohaemorrhagic Escherichia coli, Burkholderia pseudomallei, Burkholderia dolosa, Burkholderia gladioli, Burkholderia glumae, Burkholderia mallei, Burkholderia multivorans, Burkholderia oklahomensis, Burkholderia pseudomallei, Burkholderia vietnamiensis, Yersinia pestis, Erwinia amylovora, Dickeya dadanthii, Erwinia carotovora, Erwinia papayae, Erwinia psidii, Erwinia pyrifoliae, Erwinia tracheiphila, Dickeya solani, or Chlamydia trachomatis.

In some embodiments, the gram-negative bacteria is Pseudomonas syringae pv. maculicola, Pseudomonas syringae pv. tomato, Pseudomonas syringae pv. glycinea, Pseudomonas syringae pv. lachrymans, Pseudomonas syringae pv. apii, Pseudomonas syringae pv. phaseolicola, Pseudomonas syringae pv. aceris, Pseudomonas syringae pv. actinidiae, Pseudomonas syringae pv. aesculi, Pseudomonas syringae pv. aptata, Pseudomonas syringae pv. coronafaciens, Pseudomonas syringae pv. japonica, Pseudomonas syringae pv. morsprunorum, Pseudomonas syringae pv. tabaci, Pseudomonas syringae pv. syringae, Pseudomonas amygdali pv. aesculi, Pseudomonas amygdali pv. amygdali, Pseudomonas amygdali pv. ciccaronei, Pseudomonas amygdali pv. dendropanacis, Pseudomonas amygdali pv. eriobotryae, Pseudomonas amygdali pv. glycinea, Pseudomonas amygdali pv. hibisci, Pseudomonas amygdali pv. lachrymans, Pseudomonas amygdali pv. mellea, Pseudomonas amygdali pv. mori, Pseudomonas amygdali pv. morsprumorum, Pseudomonas amygdali pv. myricae, Pseudomonas amygdali pv. phaseolicola, Pseudomonas amygdali pv. photiniae, Pseudomonas amygdali pv. sesami, Pseudomonas amygdali pv. tabaci, Pseudomonas amygdali pv. ulmi, Pseudomonas coronafaciens pv. atropurpurea, Pseudomonas coronafaciens pv. coronafaciens, Pseudomonas coronafaciens pv. garcae, Pseudomonas coronafaciens pv. oryzae, Pseudomonas coronafaciens pv. porri, Pseudomonas coronafaciens pv. striafaciens, Pseudomonas coronafaciens pv. zizaniae, Pseudomonas helianthi pv. helianthi, Pseudomonas helianthi pv. tagetis, Pseudomonas savastanoi pv. fraxini, Pseudomonas savastanoi pv. nerii, Pseudomonas savastanoi. pv. oleae, Pseudomonas savastanoi pv. phaseolicola, Pseudomonas savastanoi pv. savastanoi, Pseudomonas tomato pv. antirrhini, Pseudomonas tomato pv. apii, Pseudomonas tomato pv. berberidis, Pseudomonas tomato pv. delphinii, Pseudomonas tomato pv. lachrymans, Pseudomonas tomato pv. maculicola, Pseudomonas tomato pv. morsprunorum, Pseudomonas tomato pv. passiflorae, Pseudomonas tomato pv. persicae, Pseudomonas tomato pv. philadelphi, Pseudomonas tomato pv. primulae, Pseudomonas tomato pv. ribicola, Pseudomonas tomato pv. tomato, Pseudomonas tomato pv. viburni, Pseudomonas viridiflava pv. primulae, Pseudomonas viridiflava pv. ribicola, Pseudomonas viridiflava pv. viridiflava, Pseudomonas marginalis pv. alfalfa, Pseudomonas marginalis pv. marginalis, Pseudomonas marginalis. pv. pastinacae, Xanthomonas campestris pv. armoraciae, Xanthomonas campestris pv. begonia, Xanthomonas campestris pv. begoniae, Xanthomonas campestris pv. campestris, Xanthomonas campestris pv. cannabis, Xanthomonas campestris pv. carota, Xanthomonas campestris pv. corylina, Xanthomonas campestris pv. dieffenbachiae, Xanthomonas campestris pv. glycines, Xanthomonas campestris pv. graminis, Xanthomonas campestris pv. hederae, Xanthomonas campestris pv. hyacinthi, Xanthomonas campestris pv. juglandis, Xanthomonas campestris pv. malvacearum, Xanthomonas campestris pv. musacearum, Xanthomonas campestris pv. mangiferaeindicae, Xanthomonas campestris pv. mori, Xanthomonas campestris pv. nigromaculans, Xanthomonas campestris pv. pelargonii, Xanthomonas campestris pv. phaseoli, Xanthomonas campestris pv. poinsettiicola, Xanthomonas campestris pv. pruni, Xanthomonas campestris pv. raphanin, Xanthomonas campestris pv. sesame, Xanthomonas campestris pv. tardicrescens, Xanthomonas campestris pv. translucens, Xanthomonas campestris pv. vesicatoria, Xanthomonas campestris pv. viticola, Xanthomonas oryzae pv. oryzae, Xanthomonas oryzae pv. oryzicola, S Salmonella Choleraesuis, Salmonella Dublin, Salmonella Enteritidis, Salmonella Gallinarum, Salmonella Hadar, Salmonella Heidelberg, Salmonella Infantis, Salmonella paratyphi, Salmonella typhi, Salmonella typhimurium, enterotoxigenic Escherichia coli, enteropathogenic Escherichia coli, enteroinvasive Escherichia coli, enteroaggregative Escherichia coli, Burkholderia gladioli pv. gladioli, Burkholderia gladioli pv. allicola, or Burkholderia gladioli pv. agaricicola.

### Methods of Preparation

In another aspect, provided is a method of preparing a compound disclosed herein, or a stereoisomer or tautomer thereof, or a salt of any of the foregoing, comprising converting a compound of formula (II),

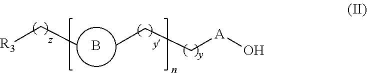

or a stereoisomer or tautomer thereof, or a salt of any of the foregoing, to the compound of formula (I),

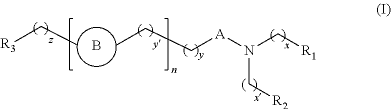

or a stereoisomer or tautomer thereof, or a salt of any of the foregoing, wherein x, x′, R₁, R₂, R₃, A, B, y, y′, z and n are as disclosed herein.

In one aspect, provided is method of preparing a compound, or a stereoisomer or tautomer thereof, or a salt of any of the foregoing, wherein the compound is selected from the group consisting of:

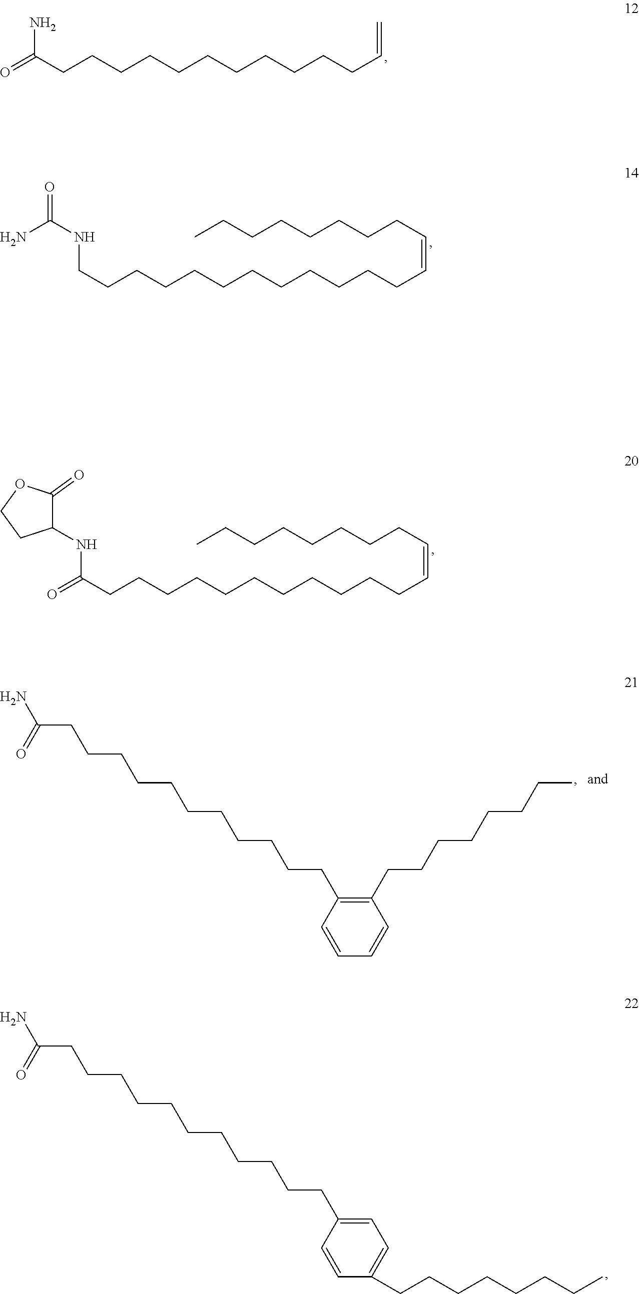 [SMILES: C=CCCCCCCCCCCCC(N)=O ; CCCCCCCC/C=C\CCCCCCCCCCCCNC(N)=O ; CCCCCCCC/C=C\CCCCCCCCCCCC(=O)NC1CCOC1=O ; CCCCCCCCc1ccccc1CCCCCCCCCCCC(N)=O ; CCCCCCCCc1ccc(CCCCCCCCCCCC(N)=O)cc1]

or a stereoisomer or tautomer thereof, or a salt of any of the foregoing.

In another aspect, provided is a method of preparing a compound of formula (IA), or a stereoisomer or tautomer thereof, or a salt of any of the foregoing,

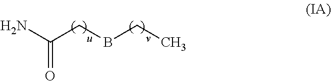

wherein:
B is phenylene, and u and v are each independently an integer of at least 1.

In some embodiments, provided a method of preparing a compound shown in Table 1, or a stereoisomer or tautomer thereof, or a salt of any of the foregoing.

In some embodiments, one or more steps of a preparation method disclosed herein comprise acylation, condensation, reduction, protection, deprotection, halogenation, substitution, hydrolysis, and/or amidation.

Representative schemes for preparing the compounds disclosed herein are provided below.

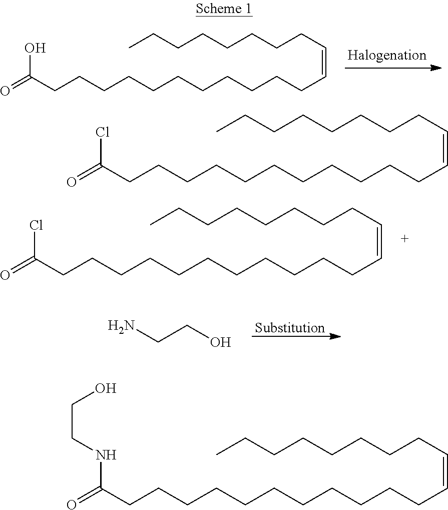

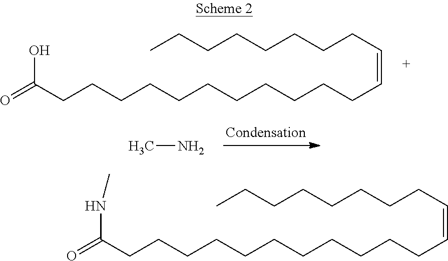

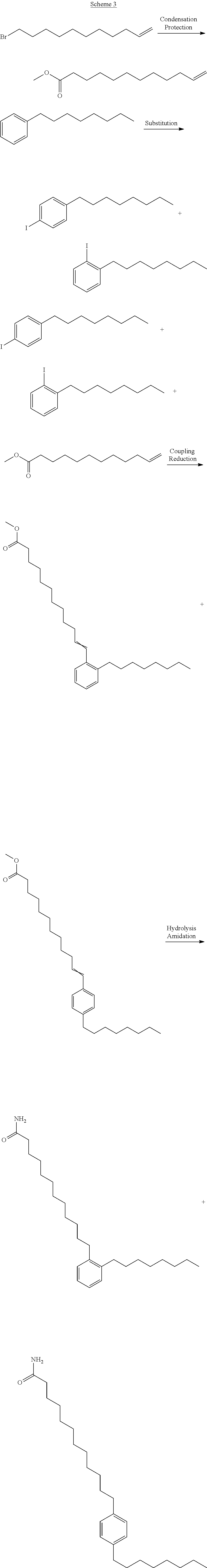

Compounds of formula (I) or any related formula described herein can be synthesized using standard synthetic techniques known to those of ordinary skill in the art. Compounds of the present disclosure can be synthesized using the general synthetic procedures set forth in the schemes provided above and examples provided below.

### EXAMPLES

### Synthetic Examples

Compounds disclosed herein can be prepared from commercially available starting materials and preparing methods described herein. The following examples serve to illustrate the compounds disclosed herein and the preparation processes thereof. These examples and preparation processes described below should not be considered as limiting the scope of the present disclosure.

The structures of the compounds in the present disclosure were confirmed by ¹H NMR. All of the compounds or intermediates in the synthetic steps were purified by column chromatography, or preparative reverse-phase HPLC unless otherwise specified. The reaction process can be detected by thin layer chromatography, and the commonly used elution systems in the purification stage were petroleum ether/ethyl acetate and dichloromethane/methanol.

### Example S1: Synthesis of (E)-13-docosenamide (Compound T3SI-2)

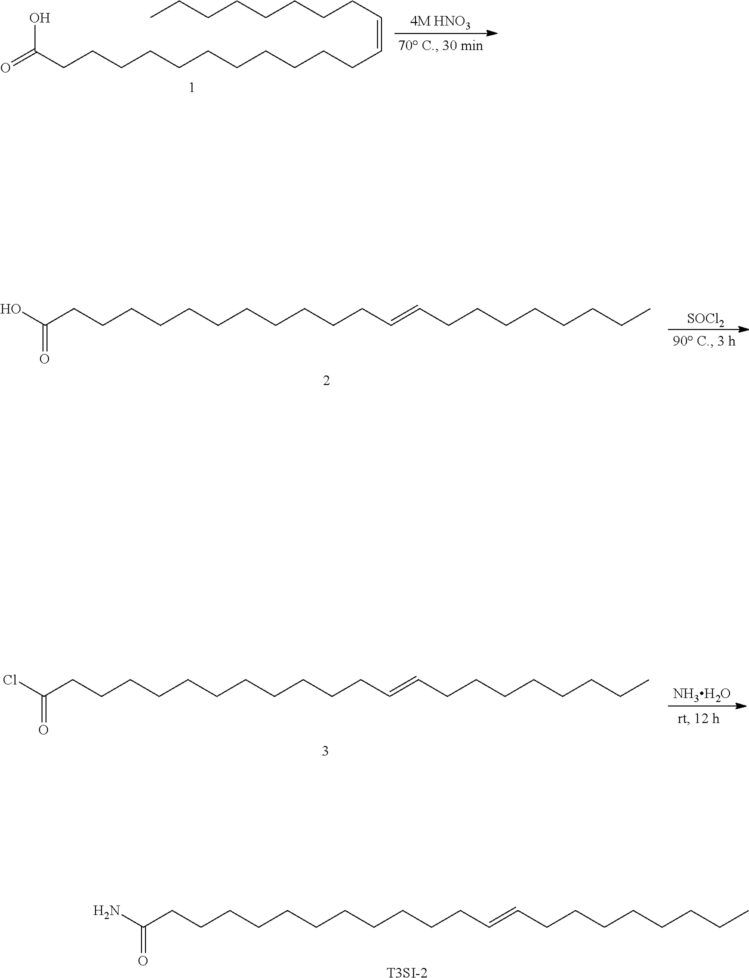

Step 1: Synthesis of brassidic (trans-13-docosenoic) acid (compound 2). 200 mg of erucic acid was added to a 50 ml round-bottom flask and heated to 70° C. under nitrogen. Then 0.72 ml of 6.0 M HNO₃ and 1.06 ml of 2.0 M NaNO₂ (4 mole % HNO₂) were added with vigorously stirring. After stirring for 30 min, the reaction was cooled to room temperature and dissolved in 100 ml ethyl ether. The organic layer was washed with water, dried with sodium sulfate. The ether was removed and the product was recrystallized from 200 ml of 95% ethanol at 4° C. 130 mg white solid product was acquired.

¹H NMR (400 MHz, Chloroform-d) δ 5.38 (dd, J=4.6, 2.8 Hz, 2H), 2.35 (t, J=7.5 Hz, 2H), 2.07-1.86 (m, 4H), 1.64 (q, J=7.4 Hz, 2H), 1.46-1.07 (m, 28H), 0.88 (t, J=6.8 Hz, 3H).

Step 2: Synthesis of (E)-13-docosenamide (Compound T3SI-2). 85 mg of brassidic acid (compound 2) (0.25 mmol) was dissolved in thionyl chloride (1 mL) and stirred at 90° C. for 3 h. The reaction was cooled to room temperature and excess thionyl chloride was removed to yield brassidic acid chloride (compound 3). The chloride was dissolved in dichloromethane (0.5 ml) and then added dropwise into concentrated ammonia (25%, 15 mL) with vigorously stirring. The reaction was stirred at room temperature for 12 h. The reaction mixture was extracted three times with dichloromethane, and the combined organic layer is dried with sodium sulfate. After removing the dichloromethane under reduced pressure, the crude product was purified by column chromatography on silica gel in dichloromethane/MeOH (50:1) to yield (E)-13-docosenamide (Compound T3SI-2).

¹H NMR (400 MHz, Chloroform-d) δ 5.53 (s, 2H), 5.38 (td, J=3.7, 1.8 Hz, 2H), 2.26-2.18 (m, 2H), 1.96 (q, J=6.3 Hz, 4H), 1.67-1.59 (m, 2H), 1.34-1.23 (m, 28H), 0.88 (t, J=6.8 Hz, 3H).

### Example S2: Synthesis of 13Z,16Z-docosadienoic acid amide (Compound T3SI-10)

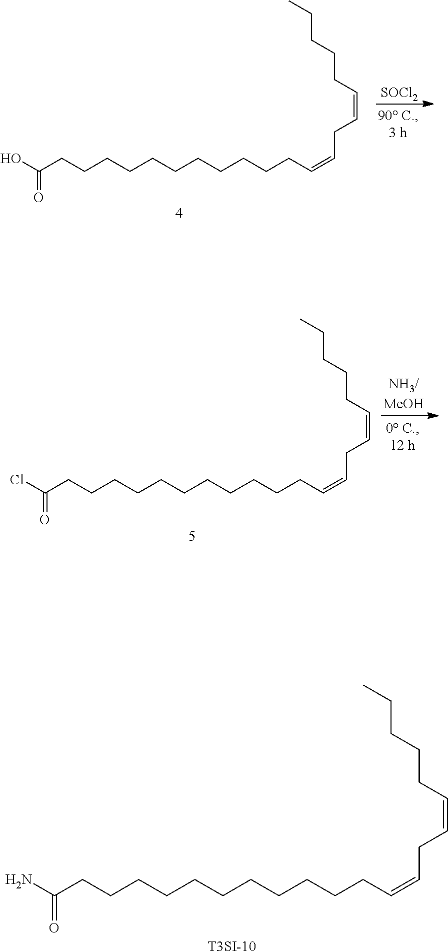

Step 1: Synthesis of 13Z,16Z-docosadienoic acid amide (Compound T3SI-10). 10 mg of 13Z,16Z-docosadienoic acid (compound 4) (0.03 mmol) was dissolved in thionyl chloride (0.5 mL) and stirred at 90° C. for 3 h. The reaction was cooled to room temperature and excess thionyl chloride was removed to yield 13Z,16Z-docosadienoic acid chloride (compound 5). The chloride was dissolved in dichloromethane (5 ml) to which 2.0 M NH₃ solution in MeOH (1 ml) were added dropwise with vigorously stirring at 0° C. The reaction was stirred at 0° C. temperature for 12 h. After removing the solvent under reduced pressure, the crude product was purified by column chromatography on silica gel in dichloromethane/MeOH (50:1) to yield 13Z,16Z-docosadienoic acid amide (Compound T3SI-10).

¹H NMR (400 MHz, Chloroform-d) δ 5.60-5.45 (m, 2H), 5.45-5.26 (m, 4H), 2.78 (t, 2H), 2.22 (t, J=7.6 Hz, 2H), 2.06 (dp, J=21.1, 7.3 Hz, 4H), 1.64 (q, J=7.6 Hz, 2H), 1.28 (d, J=14.9 Hz, 22H), 1.01-0.93 (m, 3H). 5.24 (4H, m), methylene protons between two olefinic bonds at dH 2.68 (2H, t like, J 1/4 6:5 Hz), a-methylene protons at dH 2.09 (2H, t, J 1/4 7:5 Hz), and methyl protons at dH 0.81 (3H, t, J 1/446:6 Hz)

### Example S3: Synthesis of 13Z,16Z,19Z-docosatrienoic acid amide (Compound T3SI-11)

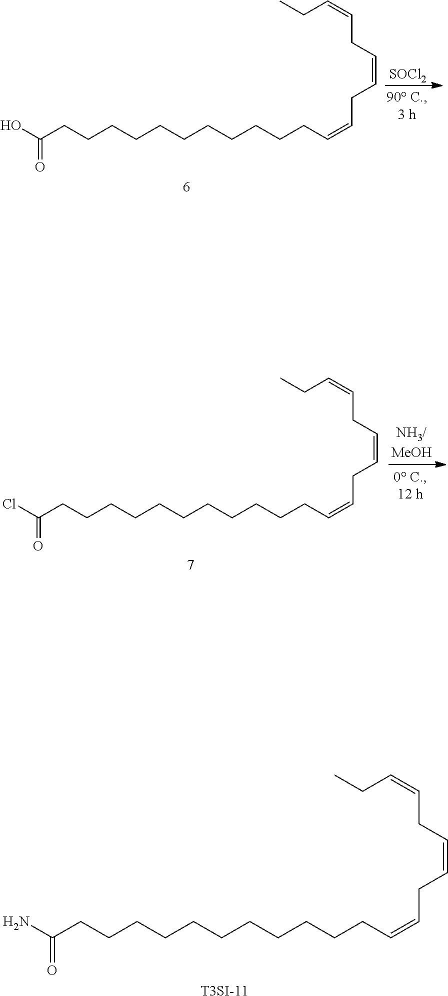

Step 1: Synthesis of 13Z,16Z,19Z-docosatrienoic acid amide (Compound T3SI-11). 10 mg of 13Z,16Z,19Z-docosatrienoic acid (compound 6) (0.03 mmol) was dissolved in thionyl chloride (0.5 mL) and stirred at 90° C. for 3 h. The reaction was cooled to room temperature and excess thionyl chloride was removed to yield 13Z,16Z,19Z-docosatrienoic acid chloride (compound 7). The chloride was dissolved in dichloromethane (5 ml) to which 2.0 M NH₃ solution in MeOH (1 ml) were added dropwise with vigorously stirring at 0° C. The reaction was stirred at 0° C. temperature for 12 h. After removing the solvent under reduced pressure, the crude product was purified by column chromatography on silica gel in dichloromethane/MeOH (50:1) to yield 13Z,16Z,19Z-docosatrienoic acid amide (Compound T3SI-11).

¹H NMR (400 MHz, Chloroform-d) δ 5.54 (m, 2H), 5.46-5.25 (m, 6H), 2.80 (t, 4H), 2.23 (t, 2H), 2.10-1.97 (m, 4H), 1.66 (m, 2H), 1.28 (d, 16H), 1.01-0.93 (m, 3H).

### Example S4: Synthesis of 13-Tetradecenoic acid amide (Compound T3SI-12)

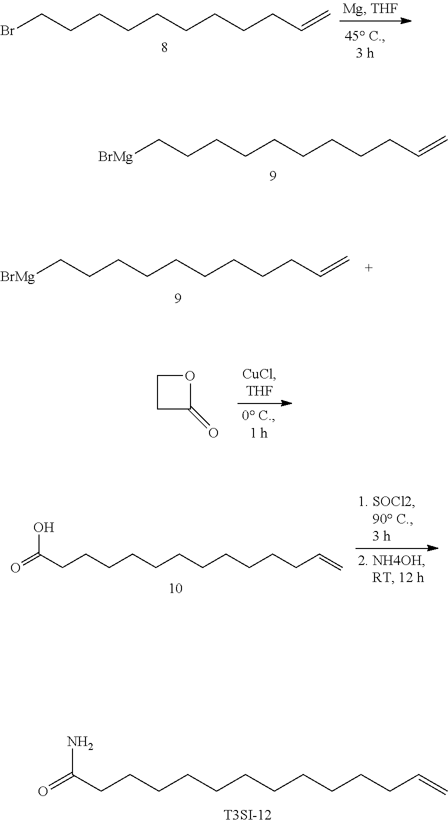

Step 1: Synthesis of 13-Tetradecenoic acid (Compound 10). 10-Undecenyl bromide (compound 8) (225 mg, 0.95 mmol, 1.0 equiv) was added dropwise to freshly scoured Mg turnings (500 mg, 2.05 mmol, 2.15 equiv) in 2 mL of dry THF with vigorously stirring under nitrogen. After 3 h at 45° C., the Grignard solution (compound 9) was cooled and transferred to 2 mL of dry THF at 0° C. with CuCl₍ₐₙₕ₎ (1.6 mg, 0.016 mmol). Then β-Propiolactone (0.5 mL, 0.79 mmol, 0.83 equiv) was added slowly via syringe at 0° C. After stirring 1 h at 0° C., the reaction was quenched with 0.5 ml of 3 N HCl and extracted two times with EtOAc. The combined organic layer was dried over Na₂SO₄, and removed under reduced pressure. The crude product was purified by column chromatography on silica gel in EtOAc/petroleum ether (1:5). 150 mg white waxy solid 13-Tetradecenoic acid (Compound 10) was acquired.

¹H NMR (400 MHz, Chloroform-d) δ 5.81 (ddt, J=16.9, 10.3, 6.6 Hz, 1H), 5.00-4.83 (m, 2H), 2.35 (m, 2H), 2.04 (q, J=7.2 Hz, 2H), 1.62 (q, J=7.2 Hz, 2H), 1.44-1.03 (m, 16H).

Step 2: Synthesis of 13-Tetradecenoic acid amide (Compound T3SI-12). 50 mg of 13-Tetradecenoic acid (compound 10) (0.22 mmol) was dissolved in thionyl chloride (1 mL) and stirred at 90° C. for 3 h. The reaction was cooled to room temperature and excess thionyl chloride was removed to yield 13-Tetradecenoic acid chloride. The chloride was dissolved in dichloromethane (1 ml) and then added dropwise into concentrated ammonia (25%, 15 mL) with vigorously stirring. The reaction was stirred at room temperature for 12 h. The reaction mixture was extracted three times with dichloromethane, and the combined organic layer is dried with sodium sulfate. After removing the dichloromethane under reduced pressure, the crude product was purified by column chromatography on silica gel in dichloromethane/MeOH (50:1) to yield 13-Tetradecenoic acid amide 43 mg (Compound T3SI-12).

¹H NMR (400 MHz, Chloroform-d) δ 5.81 (ddt, J=16.9, 10.2, 6.6 Hz, 1H), 5.53 (s, 2H), 5.05-4.83 (m, 2H), 2.23 (t, J=7.6 Hz, 2H), 2.13-1.96 (m, 2H), 1.64 (t, J=7.3 Hz, 2H), 1.45-1.12 (m, 16H).

### Example S5: Synthesis of N-(13Z)-13-Docosen-1-ylurea (Compound T3SI-14)

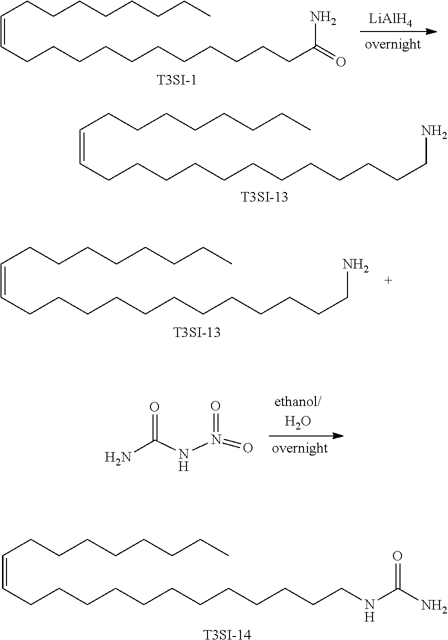

Step 1: Synthesis of 13(Z)-Docosen-1-amine (Compound T3SI-13). LiAlH₄ (130 mg) was added to dry THF, and the 99% erucamide (Compound T3SI-1) (337 mg) was added slowly. The reaction was stirred at RT overnight. Then a saturated solution of NaK tartrate (2 ml) was added. The mixture was extracted twice with ether (10 ml). The combined organic layer was washed with NaK tartrate solution and then with water. Solvent was removed under reduced pressure. The crude product was purified by column chromatography on silica gel in dichloromethane/MeOH (10:1) to yield 13(Z)-Docosen-1-amine (Compound T3SI-13).

¹H NMR (400 MHz, Chloroform-d) δ 5.35 (t, J=4.8 Hz, 2H), 2.81-2.60 (m, 4H), 2.01 (q, J=6.4 Hz, 4H), 1.47 (p, J=7.1 Hz, 2H), 1.38-1.19 (m, 30H), 0.88 (t, J=6.7 Hz, 3H).

Step 2: Synthesis of N-(3Z)-13-Docosen-1-ylurea (Compound T3SI-14). 160 mg of 13(Z)-Docosen-1-amine (Compound T3SI-13) and 100 mg of nitrourea were added into 90% ethanol with vigorous stirring. The reaction was maintained at RT overnight. The solvent was removed under reduced pressure and the crude product was purified by column chromatography on silica gel in dichloromethane/MeOH (20:1) to yield N-(13Z)-13-Docosen-1-ylurea (Compound T3SI-14).

¹H NMR (400 MHz, Chloroform-d) δ 5.35 (t, J=4.8 Hz, 2H), 4.83 (s, 1H), 4.17 (s, 2H), 3.14 (t, J=7.2 Hz, 2H), 2.01 (q, J=6.4 Hz, 4H), 1.50 (t, J=7.1 Hz, 2H), 1.40-1.06 (m, 30H), 0.88 (t, J=6.7 Hz, 3H).

### Example S6: Synthesis of N-methyl-13(Z)-Docosenamide (Compound T3SI-15)

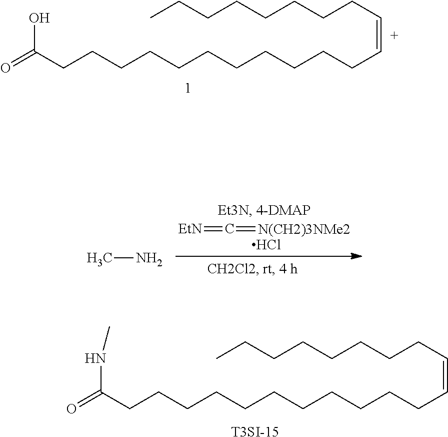

Step 1: Synthesis of N-methyl-13(Z)-Docosenamide (Compound T3SI-15). Cis-13-docosenoic acid (Compound 1) (1.0 mmol), MeNH2·HCl (1.20 mmol), Et3N (3.6 mmol, 3.6 equivalents), N, N-dimethylaminopyridine (1.0 mmol, 1.2 equivalents) and 1-(3-dimethylaminopropyl)-3-ethylcarbodiimide hydrochloride (1.2 mmol, 1.2 equivalents) were stirred in CH2Cl2 (4 mL). After 2 hours at room temperature, NaHCO₃ aqueous was added to quench the reaction. The reaction mixture was extracted three times with CH2Cl2, and the combined organic layer was removed under reduced pressure. The crude product was purified by column chromatography on silica gel in dichloromethane/MeOH (50:1) to yield N-methyl-13(Z)-Docosenamide (Compound T3SI-15).

¹H NMR (400 MHz, Chloroform-d) δ 5.58 (s, 1H), 5.35 (t, J=4.8 Hz, 2H), 2.81 (d, J=3.5 Hz, 3H), 2.18 (t, J=7.6 Hz, 2H), 2.01 (q, J=6.4 Hz, 4H), 1.62 (p, J=7.2 Hz, 2H), 1.28 (dd, J=12.1, 5.6 Hz, 28H), 0.94-0.80 (m, 3H).

### Example S7: Synthesis of N-ethyl-13(Z)-Docosenamide (Compound T3SI-16)

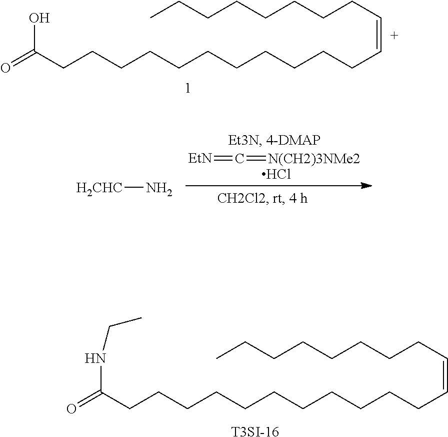

Step 1: Synthesis of N-ethyl-13(Z)-Docosenamide (Compound T3SI-16). Cis-13-docosenoic acid (Compound 1) (1.0 mmol), EtNH2·HCl (1.20 mmol), Et3N (3.6 mmol, 3.6 equivalents), N, N-dimethylaminopyridine (1.0 mmol, 1.2 equivalents) and 1-(3-dimethylaminopropyl)-3-ethylcarbodiimide hydrochloride (1.2 mmol, 1.2 equivalents) were stirred in CH2Cl2 (4 mL). After 2 hours at room temperature, NaHCO₃ aqueous was added to quench the reaction. The reaction mixture was extracted three times with CH2Cl2, and the combined organic layer was removed under reduced pressure. The crude product was purified by column chromatography on silica gel in dichloromethane/MeOH (50:1) to yield N-ethyl-13(Z)-Docosenamide (Compound T3SI-16).

¹H NMR (400 MHz, Chloroform-d) δ 5.52 (s, 1H), 5.35 (t, J=4.8 Hz, 2H), 3.29 (dt, J=11.8, 5.8 Hz, 2H), 2.16 (t, J=7.6 Hz, 2H), 2.01 (q, J=6.5 Hz, 4H), 1.62 (t, J=7.3 Hz, 2H), 1.39-1.16 (m, 28H), 1.14 (t, J=7.2 Hz, 3H), 0.93-0.83 (m, 3H).

### Example S8: Synthesis of N-methyl-Hexadecanamide (Compound T3SI-17)

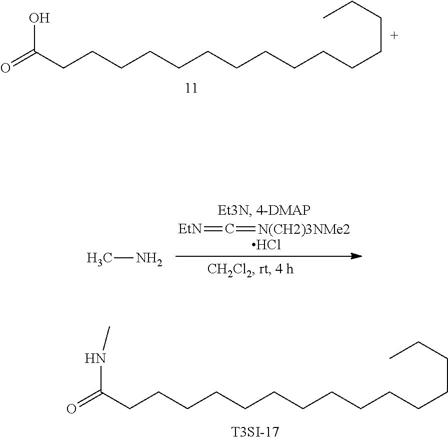

Step 1: Synthesis of N-methyl-Hexadecanamide (Compound T3SI-17). Hexadecanoic acid (Compound 11) (1.0 mmol), MeNH2·HCl (1.20 mmol), Et3N (3.6 mmol, 3.6 equivalents), N, N-dimethylaminopyridine (1.0 mmol, 1.2 equivalents) and 1-(3-dimethylaminopropyl)-3-ethylcarbodiimide hydrochloride (1.2 mmol, 1.2 equivalents) were stirred in CH2Cl2 (4 mL). After 2 hours at room temperature, NaHCO₃ aqueous was added to quench the reaction. The reaction mixture was extracted three times with CH2Cl2, and the combined organic layer was removed under reduced pressure. The crude product was purified by column chromatography on silica gel in dichloromethane/MeOH (50:1) to yield N-methyl-Hexadecanamide (Compound T3SI-17).

¹H NMR (400 MHz, Chloroform-d) δ 5.50 (s, 1H), 2.81 (d, J=4.8 Hz, 3H), 2.19-2.12 (m, 2H), 1.62 (p, J=7.6 Hz, 2H), 1.26 (d, J=10.6 Hz, 24H), 0.88 (t, J=6.8 Hz, 3H).

### Example S9: Synthesis of N-(2-hydroxyethyl)-13(Z)-Docosenamide (Compound T3SI-18)

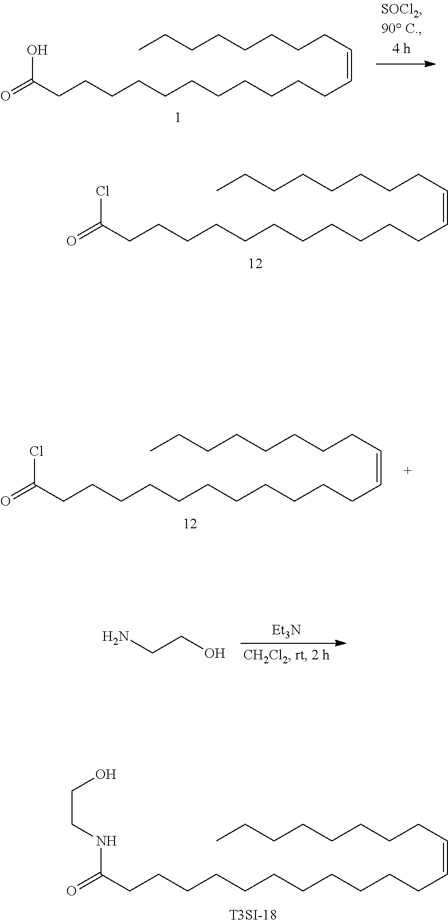

Step 1: Synthesis of N-(2-hydroxyethyl)-13(Z)-Docosenamide (Compound T3SI-18). 168 mg of erucic acid (compound 1) (0.5 mmol) was dissolved in thionyl chloride (2 mL) and stirred at 90° C. for 3 h. The reaction was cooled to room temperature and excess thionyl chloride was removed to yield erucic acid chloride (compound 12). The chloride was dissolved in dichloromethane (5 ml) and then added dropwise into a solution of 2-aminoethanol (167 mg, 0.45 mmol) and Et3N (125 μL, 0.91 mmol) in anhydrous CH2Cl2 (5 mL) with vigorously stirring. After 2 h, the solvent is evaporated and the crude was purified by column chromatography (CH₂Cl₂:MeOH=20:1) to yield N-(2-hydroxyethyl)-13(Z)-Docosenamide (Compound T3SI-18).

¹H NMR (400 MHz, Chloroform-d) δ 6.10 (s, 1H), 5.36 (dt, J=15.0, 4.2 Hz, 2H), 3.73 (t, J=4.9 Hz, 2H), 3.43 (q, J=4.8 Hz, 2H), 2.22 (t, J=7.6 Hz, 2H), 1.99 (dq, J=19.6, 6.6 Hz, 4H), 1.64 (t, J=7.3 Hz, 2H), 1.42-1.12 (m, 28H), 0.88 (t, J=6.7 Hz, 3H).

### Example S10: Synthesis of N-(2-hydroxyethyl)-Hexadecanamide (Compound T3SI-19)

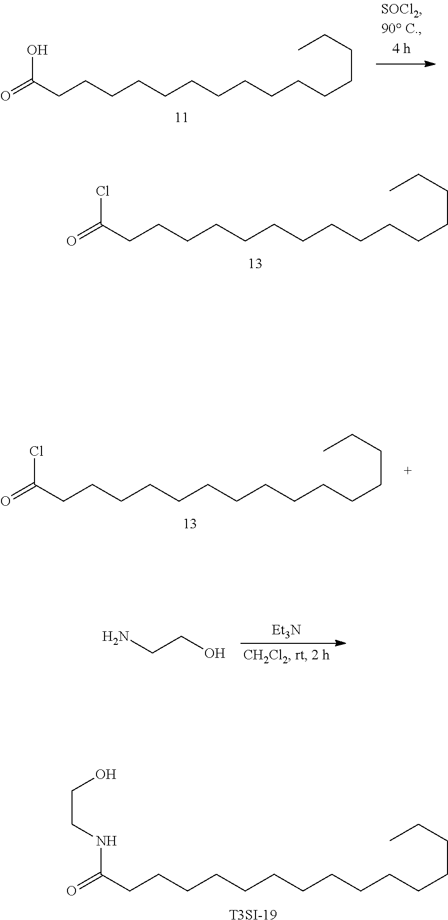

Step 1: Synthesis of N-(2-hydroxyethyl)-Hexadecanamide (Compound T3SI-19). 128 mg of Hexadecanoic acid (compound 1) (0.5 mmol) was dissolved in thionyl chloride (2 mL) and stirred at 90° C. for 3 h. The reaction was cooled to room temperature and excess thionyl chloride was removed to yield erucic acid chloride (compound 13). The chloride was dissolved in dichloromethane (5 ml) and then added dropwise into a solution of 2-aminoethanol (167 mg, 0.45 mmol) and Et3N (125 μL, 0.91 mmol) in anhydrous CH₂Cl₂ (5 mL) with vigorously stirring. After 2 h, the solvent is evaporated and the crude was purified by column chromatography (CH₂Cl₂:MeOH=20:1) to yield N-(2-hydroxyethyl)-Hexadecanamide (Compound T3SI-19).

¹H NMR (400 MHz, Chloroform-d) δ 5.96 (s, 1H), 3.73 (t, J=4.8 Hz, 2H), 3.43 (q, J=4.9 Hz, 2H), 2.21 (t, J=7.6 Hz, 2H), 1.64 (t, J=7.3 Hz, 2H), 1.25 (s, 24H), 0.88 (t, J=6.7 Hz, 3H).

### Example S11: Synthesis of N-(tetrahydro-2-oxo-3-furanyl)-13(Z)-Docosenamide (Compound T3SI-20)

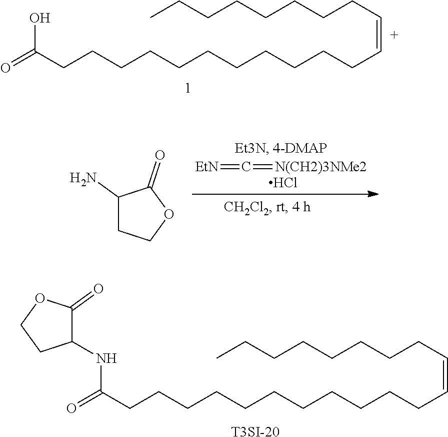

Step 1: Synthesis of N-(tetrahydro-2-oxo-3-furanyl)-13(Z)-Docosenamide (Compound T3SI-20). erucic acid (Compound 1) (1.0 mmol), L-homoserine lactone hydrochloride (1.20 mmol), Et3N (3.6 mmol, 3.6 equivalents), N, N-dimethylaminopyridine (1.0 mmol, 1.2 equivalents) and 1-(3-dimethylaminopropyl)-3-ethylcarbodiimide hydrochloride (1.2 mmol, 1.2 equivalents) were stirred in CH₂Cl₂ (4 mL). After 2 hours at room temperature, NaHCO₃ aqueous was added to quench the reaction. The reaction mixture was extracted three times with CH₂Cl₂, and the combined organic layer was removed under reduced pressure. The crude product was purified by column chromatography on silica gel in dichloromethane/MeOH (50:1) to yield N-(tetrahydro-2-oxo-3-furanyl)-13(Z)-Docosenamide (Compound T3SI-20).

¹H NMR (400 MHz, Chloroform-d) δ 6.24 (d, J=6.6 Hz, 1H), 5.35 (t, J=4.8 Hz, 2H), 4.56 (ddd, J=11.6, 8.6, 5.9 Hz, 1H), 4.50-4.43 (m, 1H), 4.28 (ddd, J=11.3, 9.2, 5.9 Hz, 1H), 2.84 (ddd, J=12.9, 8.6, 5.9 Hz, 1H), 2.23 (td, J=13.1, 10.8, 7.1 Hz, 2H), 2.18-2.10 (m, 1H), 2.01 (q, J=6.4 Hz, 4H), 1.65 (q, J=7.4 Hz, 2H), 1.28 (dd, J=14.0, 5.0 Hz, 28H), 0.88 (t, J=6.7 Hz, 3H).

### Example S12: Synthesis of 12-(2-octylphenyl)dodecanamide (Compound T3SI-21) and 12-(4-octylphenyl)dodecanamide (Compound T3SI-22)

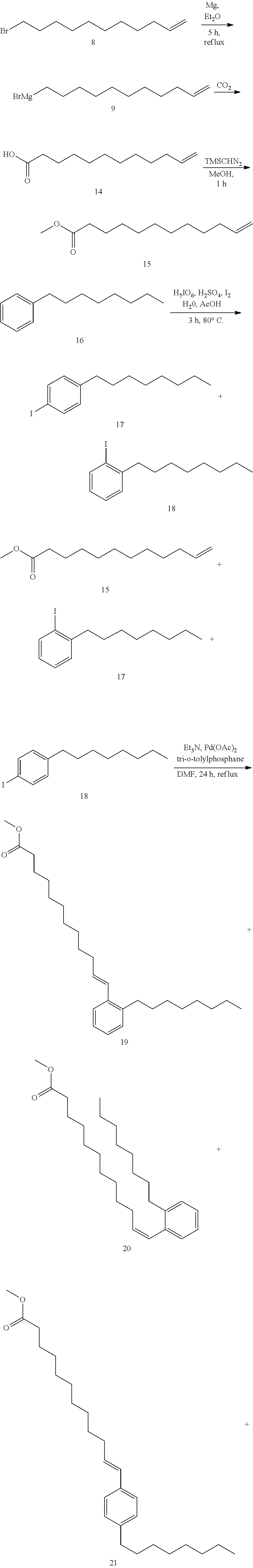

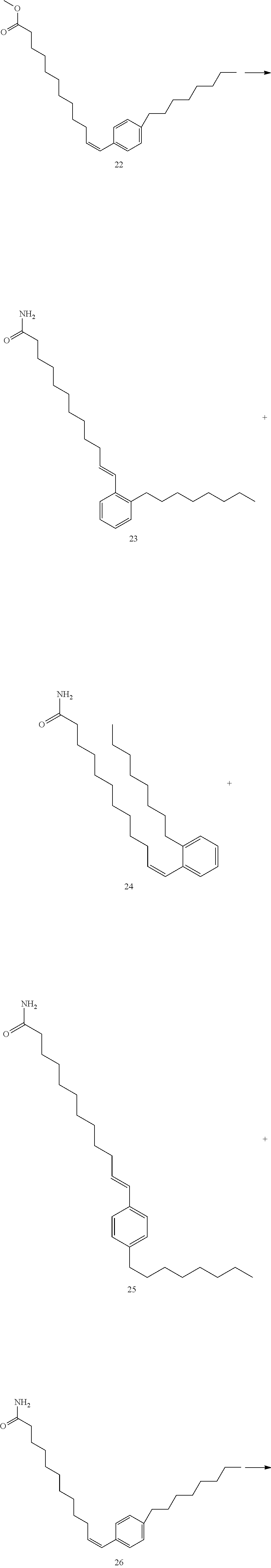 [SMILES: CCCCCCCCc1ccc(/C=C\CCCCCCCCCC(=O)OC)cc1 ; CCCCCCCCc1ccccc1/C=C/CCCCCCCCCC(N)=O ; CCCCCCCCc1ccccc1/C=C\CCCCCCCCCC(N)=O ; CCCCCCCCc1ccc(/C=C/CCCCCCCCCC(N)=O)cc1 ; CCCCCCCCc1ccc(/C=C\CCCCCCCCCC(N)=O)cc1]

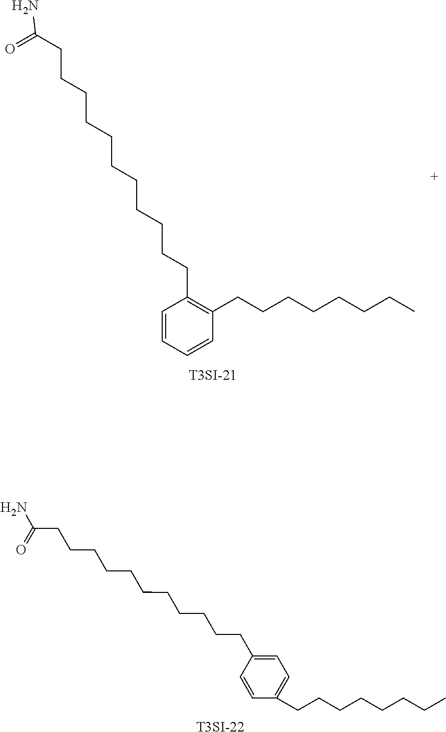

Step 1: Synthesis of 11-Dodecanoic acid (Compound 14). Freshly crushed Mg (0.5 g, 20 mmol, 2.85 eqv.) was added into dry Et₂O (2.7 ml) in a two-round neck. 11-bromo-1-undecene (1.5 ml, 7 mmol, 1.0 eqv.) was dissolved in dry Et₂O, which was slowly added to the reaction. The reaction was heated under reflux for 5 hours. The obtained Grignard solution was transferred into a new flask and was cooled to −40° C. CO₂ gas was bubbled through the solution for 2 hours. The reaction was quenched with water and the pH was adjust to 12 with NaOH to basify the product. The organic layer was discarded. The aqueous layer was acidified using 18% HCl and extracted three times with EtOAc. The crude product was purified by column chromatography on silica gel in EtOAc/petroleum ether (1:5). 11-Dodecanoic acid (Compound 14) were acquired.

1H NMR (400 MHz, Chloroform-d) δ 5.81 (ddt, J=17.0, 10.2, 6.7 Hz, 1H), 5.04-4.85 (m, 2H), 2.35 (t, J=7.5 Hz, 2H), 2.08-1.98 (m, 2H), 1.62 (q, J=7.3 Hz, 2H), 1.43-1.17 (m, 12H).

Step 2: Synthesis of methyl dodec-11-enoate (Compound 15). 200 mg (1 mmol) of 11-Dodecanoic acid (Compound 14) and 570 mg (5 mmol) TMSCHN₂ was added into dry MeOH. After stirring for 1 h at RT, the reaction was quenched with water and was extracted three times with EtOAc. The combined organic layer was dried with sodium sulfate and removed under reduced pressure. The crude product was purified by column chromatography on silica gel in EtOAc/petroleum ether (1:50) to yield dodec-11-enoate (Compound 15).

1H NMR (400 MHz, Chloroform-d) δ 5.74 (ddt, J=16.9, 10.2, 6.7 Hz, 1H), 4.94-4.79 (m, 2H), 3.60 (s, 3H), 2.23 (t, J=7.6 Hz, 2H), 1.99-1.89 (m, 2H), 1.56 (q, J=7.6 Hz, 2H), 1.37-1.11 (m, 12H).

Step 3: Synthesis of methyl 1-iodo-4-octylbenzene (Compound 17) and 1-iodo-2-octylbenzene (Compound 18). A mixture of 1-phenyloctane (1.48 g, 7.813 mmol), H₅IO₆ (356 mg, 1.56 mmol), iodine (793 mg, 3.12 mmol), acetic acid (4 mL), 98% sulfuric acid (259 mg), and deionized water (0.7 mL) in a 50 mL flask. The reaction was heated at 80° C. for about 4 h and was subsequently extracted 3 times with dichloromethane. The organic layer was washed with saturated aq. NaHCO₃ and evaporated under reduced pressure. The crude product was purified by column chromatography on silica gel using petroleum ether. Colorless viscous liquid was obtained as a mixture of 1-iodo-4-octylbenzene (Compound 17) and 1-iodo-2-octylbenzene (Compound 18).

1H NMR (400 MHz, Chloroform-d) δ 7.57 (d, J=8.2 Hz, 1H), 7.29-7.12 (m, 1H), 6.95-6.81 (m, 2H), 2.73-2.65 (m, 1H), 2.52 (d, J=7.8 Hz, 1H), 1.56 (tdd, J=13.1, 10.9, 7.3 Hz, 2H), 1.42-1.23 (m, 10H), 0.88 (td, J=6.9, 3.4 Hz, 3H).

Step 4: Synthesis of methyl (E)-12-(2-octylphenyl)dodec-11-enoate (Compound 19), methyl (Z)-12-(2-octylphenyl)dodec-11-enoate (Compound 20), methyl (E)-12-(4-octylphenyl)dodec-11-enoate (Compound 21), methyl (Z)-12-(4-octylphenyl)dodec-11-enoate (Compound 22). Triethylamine (1.03 ml, 7.3 mmol) was added into a solution of tri-o-tolylphosphane (43 mg, 0.14 mmol) and palladium diacetate (14 mg, 0.06 mmol) in dry DMF (12 ml) under nitrogen at RT. After stirring for few minutes, 95.47 mg (0.45 mmol) of dodec-11-enoate (Compound 15) was dissolved in dry DMF (3 ml) and added. Then after stirring for 15 mins, 316 mg (1 mmol) of 1-iodo-4-octylbenzene (Compound 17) and 1-iodo-2-octylbenzene (Compound 18) was dissolved in dry DMF (3 ml) and added. The reaction was heated at 90° C. for 44 h. The reaction was cooled to RT and dissolved with EtOAc (60 ml) and water (50 ml). The aqueous layer was further extracted twice with EtOAc and the combined organic layer was dried with sodium sulfate before evaporated under reduced pressure. The crude product was purified by by column chromatography on silica gel in EtOAc/petroleum ether (gradually) to yield 152 mg of the mixture of methyl (E)-12-(2-octylphenyl)dodec-11-enoate (Compound 19), methyl (Z)-12-(2-octylphenyl)dodec-11-enoate (Compound 20), methyl (E)-12-(4-octylphenyl)dodec-11-enoate (Compound 21), methyl (Z)-12-(4-octylphenyl)dodec-11-enoate (Compound 22).

1H NMR (400 MHz, Chloroform-d) δ 7.43-7.20 (m, 2H), 7.22-6.98 (m, 3H), 6.68-5.68 (m, 1H), 3.66 (s, 3H), 2.71-2.51 (m, 2H), 2.35-2.14 (m, 2H), 2.01 (d, J=1.2 Hz, 2H), 1.61 (dd, J=13.1, 7.6 Hz, 4H), 1.40-1.19 (m, 22H), 0.93-0.81 (m, 3H).

Step 5: Synthesis of (E)-12-(2-octylphenyl)dodec-11-enamide (Compound 23), (Z)-12-(2-octylphenyl)dodec-11-enamide (Compound 24), (E)-12-(4-octylphenyl)dodec-11-enamide (Compound 25) and (Z)-12-(4-octylphenyl)dodec-11-enamide (Compound 26). 152 mg of the mixture of methyl (E)-12-(2-octylphenyl)dodec-11-enoate (Compound 19), methyl (Z)-12-(2-octylphenyl)dodec-11-enoate (Compound 20), methyl (E)-12-(4-octylphenyl)dodec-11-enoate (Compound 21), methyl (Z)-12-(4-octylphenyl)dodec-11-enoate (Compound 22) was added into 15 ml of aqueous ammonia in a 50 ml three-neck round flask with mechanical stirrer and reflux condenser. The mixture was stirred at 35° C. for 72 hours and the resulting mixture was extracted with EtOAc. The organic layer was dried and removed under reduced pressure. The crude product was further purified by column chromatography on silica gel in DCM/MeOH (50:1) to yield 100 mg of the mixture of (E)-12-(2-octylphenyl)dodec-11-enamide (Compound 23), (Z)-12-(2-octylphenyl)dodec-11-enamide (Compound 24), (E)-12-(4-octylphenyl)dodec-11-enamide (Compound 25) and (Z)-12-(4-octylphenyl)dodec-11-enamide (Compound 26).

¹H NMR (400 MHz, Chloroform-d) δ 7.25 (d, J=5.0 Hz, 2H), 7.21-7.01 (m, 3H), 6.34 (d, J=15.8 Hz, 1H), 5.67-5.26 (m, 2H), 2.69-2.48 (m, 2H), 2.20 (q, J=8.1 Hz, 2H), 2.01 (d, J=1.2 Hz, 2H), 1.77-1.51 (m, 4H), 1.50-1.05 (m, 22H), 0.95-0.78 (m, 3H).

Step 6: Synthesis of 12-(2-octylphenyl)dodecanamide (Compound T3SI-21) and 12-(4-octylphenyl)dodecanamide (Compound T3SI-22). 100 mg of the mixture of (E)-12-(2-octylphenyl)dodec-11-enamide (Compound 23), (Z)-12-(2-octylphenyl)dodec-11-enamide (Compound 24), (E)-12-(4-octylphenyl)dodec-11-enamide (Compound 25) and (Z)-12-(4-octylphenyl)dodec-11-enamide (Compound 26) and 10 mg of Pd/C was added into MeOH (5 ml)/EtOAc (5 ml) under nitrogen with stirring. The nitrogen atmosphere was changed into hydrogen at −78° C. The reaction was stirred at RT overnight under hydrogen. The solvent was removed under reduced pressure and the crude products was purified with preparative reverse-phase HPLC to yield 12-(2-octylphenyl)dodecanamide (Compound T3SI-21) and 12-(4-octylphenyl)dodecanamide (Compound T3SI-22).

Compound T3SI-21: ¹H NMR (400 MHz, Chloroform-d) δ 7.08 (s, 4H), 5.41 (s, 2H), 2.56 (dd, 4H), 2.26-2.19 (m, 2H), 1.70-1.53 (m, 6H), 1.34-1.20 (m, 24H), 0.90-0.83 (m, 3H).

Compound T3SI-22: ¹H NMR (400 MHz, Chloroform-d) δ 7.10 (s, 4H), 5.53-5.32 (s, 2H), 2.57 (dd, 4H), 2.27-2.19 (m, 2H), 1.70-1.55 (m, 6H), 1.34-1.22 (m, 24H), 0.87 (m, 3H).

### Example S13: Synthesis of (5Z,8Z,11Z,14Z)-5,8,11,14-Eicosatetraenamide (Compound T3SI-23)

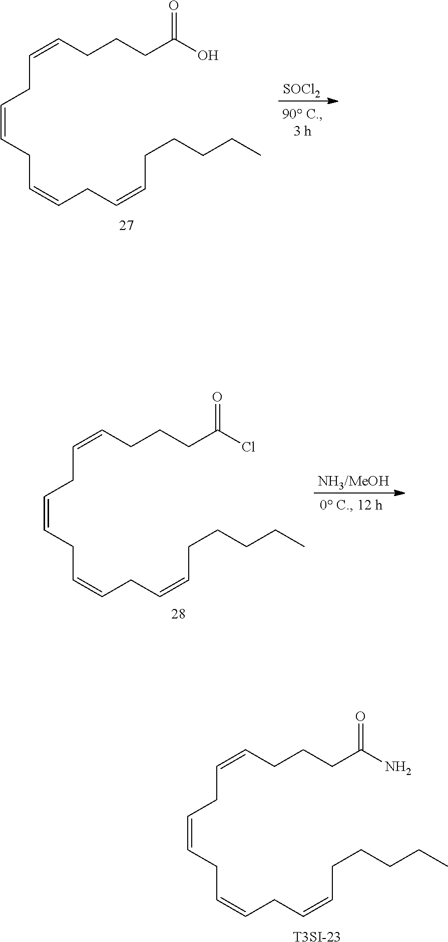

Step 1: Synthesis of (5Z,8Z,11Z,14Z)-5,8,11,14-Eicosatetraenamide (Compound T3SI-23). 50 mg of (5Z,8Z,11Z,14Z)-5,8,11,14-Eicosatetraenoic acid (compound 27) was dissolved in thionyl chloride (2 mL) and stirred at 90° C. for 3 h. The reaction was cooled to room temperature and excess thionyl chloride was removed to yield 13Z,16Z-docosadienoic acid chloride (compound 28). The chloride was dissolved in dichloromethane (5 ml) to which 2.0 M NH₃ solution in MeOH (1 ml) were added dropwise with vigorously stirring at 0° C. The reaction was stirred at 0° C. temperature for 12 h. After removing the solvent under reduced pressure, the crude product was purified by column chromatography on silica gel in dichloromethane/MeOH (50:1) to yield (5Z,8Z,11Z,14Z)-5,8,11,14-Eicosatetraenamide (Compound T3SI-23).

1H NMR (400 MHz, Chloroform-d) δ 5.81 (br s, 2H) 5.32-5.42 (m, 8H), 2.80-2.86 (m, 6H), 2.21 (t, J=8.1 Hz, 2H), 2.06-2.17 (m, 4H), 1.69-1.77 (m, 2H), 1.24-1.39 (m, 6H), 0.88 (t, J=6.8 Hz, 3H).

### Example S14: Synthesis of (Z)—N-hydroxydocos-13-enamide (Compound T3SI-24)

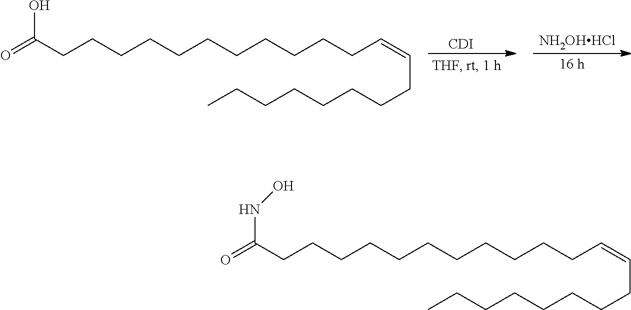

Step 1: Synthesis of (Z)—N-hydroxydocos-13-enamide (Compound T3SI-24). CDI (1.5 eq.) was added to a solution of erucic acid (1 mmol) in dry tetrahydrofuran (2 mL). Hydroxylamine hydrochloride (2 equiv) was added after stirring at RT for 1 h. The resulting mixture was stirred for 12 h. The mixture was diluted with 5% aq. KHSO4 (3 mL) and extracted with EtOAc three times. The combined organic layer was dried over sodium sulfate and removed under reduced pressure. The crude product was purified by column chromatography on silica gel in dichloromethane/MeOH (20:1).

1H NMR (400 MHz, Chloroform-d) δ 5.35 (t, J=4.9 Hz, 2H), 2.26-2.09 (m, 2H), 2.01 (q, J=6.4 Hz, 4H), 1.68 (dd, J=14.8, 7.5 Hz, 2H), 1.28 (dd, J=12.6, 5.6 Hz, 28H), 0.88 (t, J=6.7 Hz, 3H).

### Biological Examples

Erucamide is an Inhibitor of P. syringae TTSS Secretion

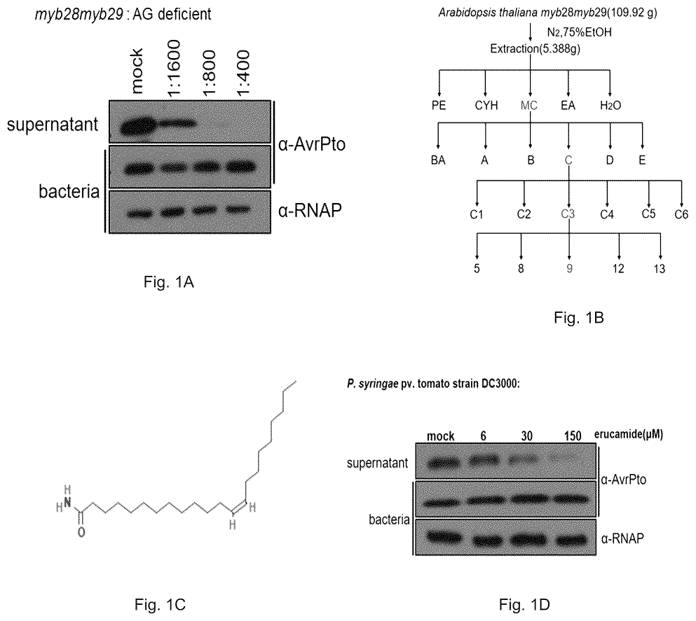

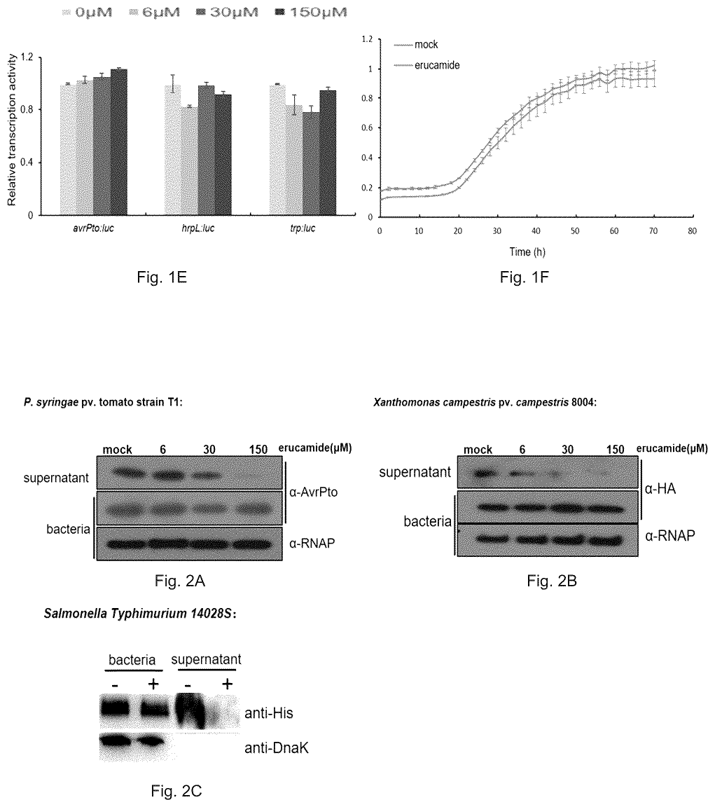

In the present work, we identified SFN, a breakdown product of aliphatic glucosinolates, preferentially inhibits P. syringae TTSS gene expression. We next observed the crude extracts of SFN-deficient mutant myb28/29 inhibited the secretion of AvrPto, a major TTSS effector, in a dose-dependent manner (FIG. 1A). We analysis AvrPto secretion of Pst DC3000 in presence of decreasing dilution of crude extracts. After 24 h treatment, the amount of secreted AvrPto at 1:1600 dilution of crude extracts was decreased to 60%. Furthermore, secreted AvrPto can not be detected in the supernatant when bacteria was incubated with increasing concentration of extracts. In contrast, the amount of intracellular AvrPto and control protein RNA polymerase beta (RNAP) were not affected by crude extracts (FIG. 1A). These results indicate that Arabidopsis plants possess metabolites containing ability of inhibiting TTSS secretion.

To identify the inhibitor of TTSS secretion, we performed bioassay-guided chemical purification. Finally, fraction 9 displayed high activity of AvrPto secretion inhibition (FIG. 1B). Using Nuclear Magnetic Resonance (NMR) and High Resolution Mass Spectrum (HRMS) and several methods, we characterize this inhibitor as erucamide (FIG. 1C). To demonstrate the inhibition of TTSS secretion by erucamide. Pst DC3000 bacteria were incubated with erucamide at indicated concentration and the efficiency of AvrPto secretion was determined. Inhibition of AvrPto secretion was observed at 6 μM, and the efficiency of AvrPto secretion was gradually decreased with increasing concentration of erucamide (FIG. 1D). To clearly rule out transcriptional effect, we determine the effects of erucamide on the transcription, we found that the transcription activity of two TTSS genes (avrPto and hrpL) and a house-keeping gene trp was similar in absence or presence of erucamide (FIG. 1E). We next test whether erucamide inhibits bacterial growth. We assayed the growth curve of Pst DC3000 with and without 150 μM erucamide, no effect on bacterial growth was observed (FIG. 1F).

### Erucamide is a Broad-Spectrum TTSS Secretion Inhibitor

TTSS is encoded by a broad range of gram-negative pathogenic bacteria with a great variety of hosts including vertebrates, plants and insects. The components and structure of different TTSS are highly conserved, so we next ask whether erucamide inhibits TTSS secretion of other gram-negative pathogenic bacteria. Effects of erucamide on inhibition of other TTSS secretion were determined, such as Pst T1, S. typhimurium, and Xcc8004. Because of the lack of endogenous antibodies, the S. typhimurium strain expressing His-tagged SopF or Xcc8004 strain expressing HA-tagged AvrAC was used to examine erucamide effects on TTSS secretion inhibition. Pst T1 bacteria, S. typhimurium bacteria or Xcc8004 bacteria were incubated with indicated concentration erucamide, the secretion of indicated effector was determined. After 24 h incubation, the secretion of AvrPto, SopF and AvrAc in erucamide-treated bacteria was significantly lower than that in untreated bacteria (FIGS. 2A-2C), indicating erucamide is a TTSS secretion inhibitor of broad spectrum.

Erucamide Inhibits TTSS Secretion and Confers Disease Resistance to P. syringae in Plants

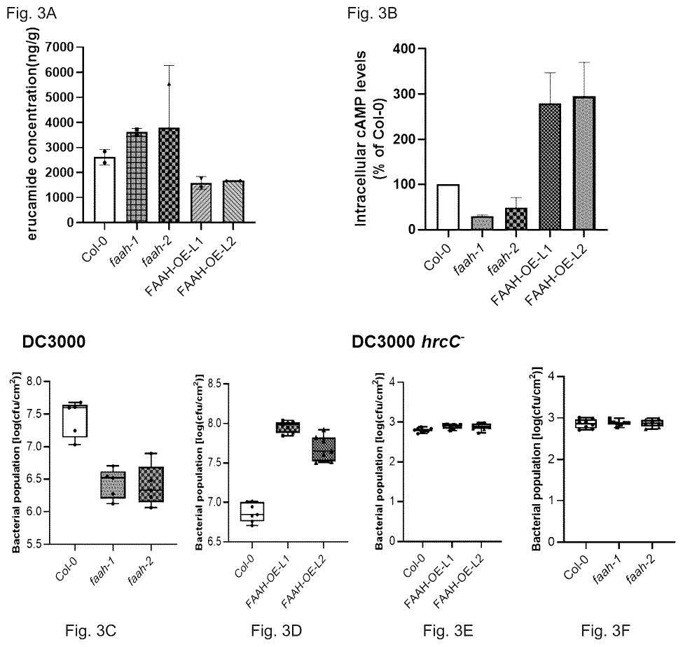

Erucamide belongs to fatty acid amides, a group of nitrogen-containing and lipid-soluble fatty acid derivatives. The biogenesis mechanism of erucamide remain poorly understood. However, plant fatty acid amide hydrolase (FAAH), an enzyme predominantly involved in N-Acylethanolamines catabolism, also can convert erucamide to erucic acid and ammonium. So we first determine erucamide content in leaves of wild type, faah mutant and FAAH overexpression plants. The concentration of erucamide in Col-0 is about 2.6 g/g, whereas 40% more erucamide was measured infaah mutants (FIG. 3A). Moreover, erucamide concentration decreased to about 1.6 mg/g in FAAH overexpression plants (FIG. 3A). We further performed CyaA injection assay to determine the levels of P. syringae effector AvrPto delivery in the faah mutant and FAAH overexpression plants. cAMP level in faah mutants was significantly less than that in wild type (FIG. 3B). Moreover, FAAH overexpression plants accumulated more cAMP than wild type (FIG. 3B). These results suggested the efficient of P. syringae TTSS secretion in erucamide-deficient plants were higher than that with more erucamide. We further investigate whether FAAH-depended fatty acid derivatives hydrolysis affects plant disease resistance to P. syringae. Pst DC3000 bacterial growth in faah-1 and faah-2 mutant plants was significantly lower than that in the wild type (FIG. 3C). Furthermore, FAAH overexpression plants supported more bacterial growth compared to wild type (FIG. 3D). These results indicated that FAAH negatively regulate plant resistance to P. syringae. Next we examined whether FAAH-mediated susceptibility to P. syringae is TTSS system-dependent. The bacterial growth of Pst DC3000 hrcC mutant with disrupted TTSS system on indicated plants was determined. We found growth of Pst DC3000 hrcC mutant was indistinguishable between wild type and faah mutants or wild type and FAAH overexpression plants (FIGS. 3E and 3F). Take together, these results support that erucamide inhibits P. syringae TTSS secretion and confer disease resistance.

### Preventive Effects of Erucamide on Plant Bacterial Diseases

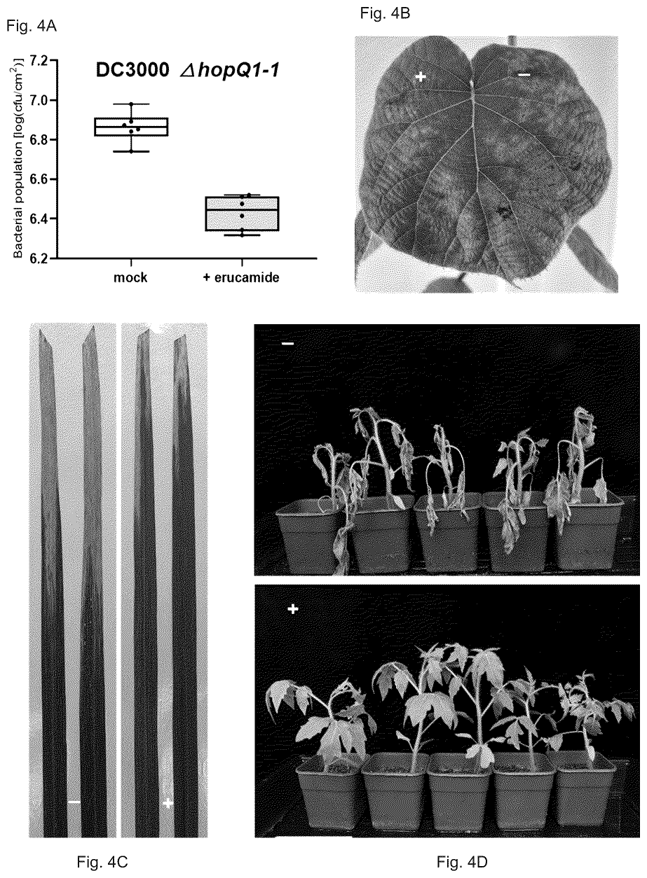

We next examine whether exogenous applications of erucamide can increase plant resistance to bacterial disease. Pst DC3000 hrcQ mutant with or without 150 μM erucamide were inoculated into leaves of N. benthamiana and bacterial growth assay was performed 3 days after inoculation. The addition of erucamide inhibits bacterial growth on plants, the bacteria population in erucamide treated plants was 5-fold less than that in untreated plants (FIG. 4A). Bacterial canker of kiwifruit, caused by P. actinidiae, is the most serious disease in kiwifruit. We further examined the preventive effects of erucamide on bacterial canker of kiwifruit. P. actinidiae bacteria with or without 1 mM erucamide were sprayed on leaves of kiwifruit, exogenous application of erucamide significantly decrease the disease symptoms in kiwifruit leaves (FIG. 4B).

We next determined whether erucamide can inhibit rice bacterial blight disease caused by Xanthomonas oryzae pv. oryzae (Xoo). Xoo isolate P6 with 1 mM erucamide or solvent were coinoculated onto leave of 2-month-old field-grown rice by leaf-clipping method. We showed that the lesion length were significantly shorter in leaves treated by erucamide than these treated by solvent (FIG. 4C). R. solanacearum is a soil-borne bacterial pathogen that is able to cause disease in more than 250 plant species, such as tomato, potato and pepper. Similar to other Gram-negative bacterial pathogens, R. solanacearum requires TTSS to secret effector proteins inside host cells and cause disease. To determine the effect of erucamide on R. solanacearum virulence, R. solanacearum with 1 mM erucamide or solvent were inoculated into tomato roots upon soil drenching. Compared with the solvent group, treatment with erucamide significantly decreased disease symptoms (FIG. 4D). These results suggested erucamide has preventive effects on plant bacterial disease.

Erucamide Inhibits P. syringe TTSS Injectisome Assembly

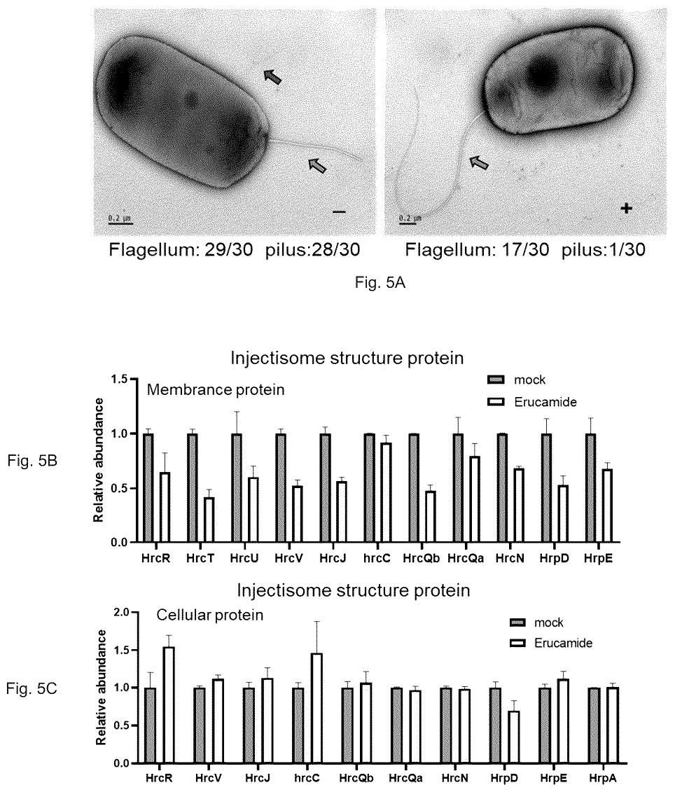

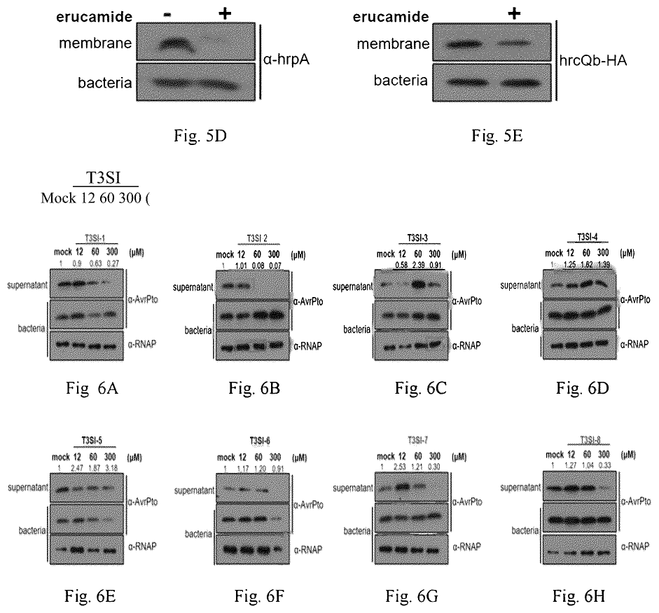

The central element of TTSSs is the injectisome, a multiprotein structure that deliver bacterial effector to host cells. In order to investigate the effect of erucamide on the injectisome, we examined injectisome also named Hrp pilus of P. syringe grown in the presence and absence of eruamide using transmission electron microscopy. In solvent treatment group, almost all the bacteria have well assembled flagella and Hrp pili, but in erucamide treatment group, only 3.3% bacteria had Hrp pilus and 56.7% bacteria had flagella (FIG. 5A). This indicated that erucamide significantly inhibited Hrp pilus assembly and slightly blocked flagella production.

To test whether the inhibition of Hrp pilus assembly actually resulted from a reduction in either cellular levels or localization of injectisome structure protein to the bacteria membrane, we performed quantitative proteomics to determine abundance of injectisome structure protein in cellular and membrane protein in presence or absence of erucamide. The results showed that erucamide treatment reduced most injectisome structure protein accumulation in membrane (FIG. 5B), but the amount of these protein in total protein was not altered by erucamide (FIG. 5C). To confirm the result from quantitative proteomics, cellular protein (bacteria) and membrane protein (membrane) were extracted from P. syringe grown with or without 0.2 mM erucamide, the abundance of HrpA and HrcQb were determined with indicated antibody. Similarly, erucamide treatment inhibited HrpA and HrcQb accumulation in membrane but did not change the levels of these two proteins in cellular (FIGS. 5D and 5E). These results indicated that erucamide inhibited TTSS secretion by disrupting injectisome assembly.

Effects of Erucamide Analogs on P. syringe TTSS Secretion

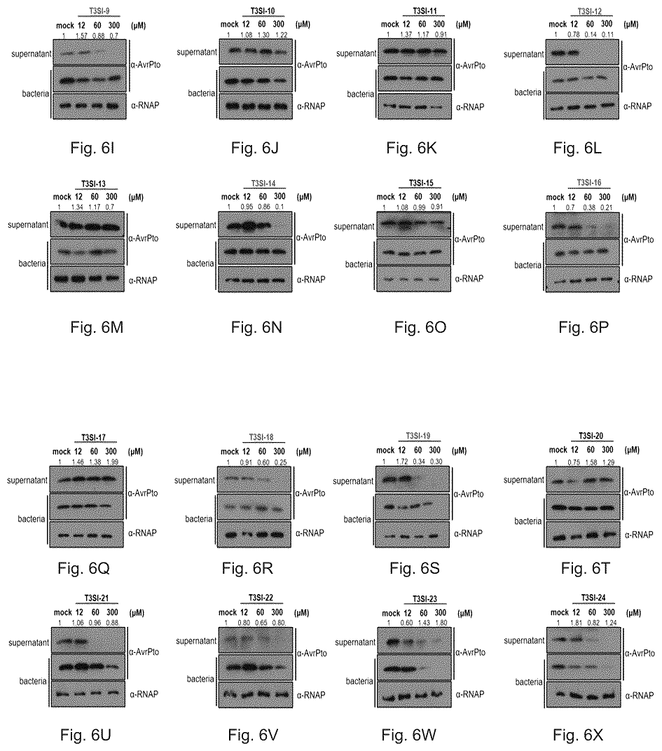

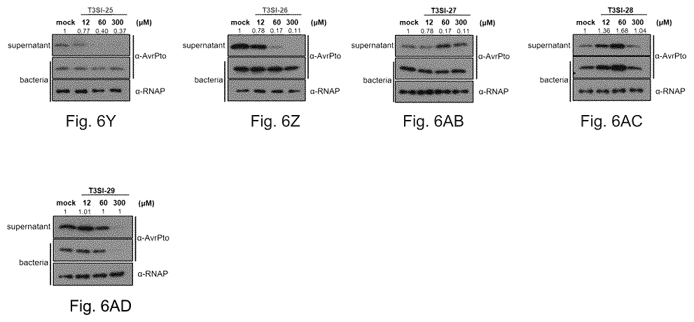

We designed and synthesized 24 erucamide analogs and analyzed their inhibitory activity on P. syringe TTSS secretion. Pst DC3000 was grown in presence of decreasing dilution of indicated analog or solvent and the efficiency of AvrPto secretion was determined. T3SI-1, T3SI-2, T3SI-7, T3SI-8, T3SI-9, T3SI-12, T3SI-14, T3SI-16, T3SI-18, T3SI-19, T3SI-25 and T3SI-26 effectively inhibited AvrPto secretion in Pst DC3000 (FIGS. 6A, 6B, 6G, 6H, 6I, 6L, 6N, 6P, 6R, 6S, 6Y and 6Z), while T3SI-5, T3SI-6, T3SI-21, T3SI-22, T3SI-23, T3SI-24 and T3SI-29 treatment decrease the abundance of AvrPto in total protein (FIGS. 6E, 6F, 6U, 6V, 6W, 6X and 6AD), other analogs were not altered neither the efficient of AvrPto nor the accumulation of AvrPto in bacteria.

### Methods

### Infection and Bacterial Growth Assay

Pst DC3000 or Pst DC3000 hopQ1-1⁻ mutant were cultured overnight at 28° C. in King's B medium containing appropriate antibiotics. Bacteria were harvested, washed twice by sterile water and diluted with sterile water or 150 μM erucamide solution to a final density OD₆₀₀=0.001 (Pst DC3000) and OD₆₀₀=0.00001 (Pst DC3000 hopQ1-1⁻). Fully expanded and healthy leaves of 6-week post-germination N. benthamiana were infiltrated with Pst DC3000 hopQ1-1 bacterial suspensions, while fully expanded and healthy leaves of 4-week post-germination A. thaliana Col-0 were infiltrated with Pst DC3000 suspensions. Bacterial populations in leaves were determined 3 days after inoculation as described previously.

For R. solanacearum soil-drenching inoculation, strain GMI 1000 was cultured overnight at 28° C. in complete BG liquid medium. Bacteria were harvested and resuspended with sterile water containing erucamide or solvent to a final density OD600=0.1. Four-to-five-week old tomatoes were inoculated by soil drenching. 50 mL of inoculum of strain was used to soak each pot of plants. Symptoms gradually appeared from three to four days after inoculation.

For P. syringae pv. actinidiae brushing inoculation, strain was cultured overnight at 28° C. in LB medium and resuspended in water containing erucamide or solvent at an OD600 of 0.5. The back side of kiwifruit leaves were inoculated by brushing with suspension containing 0.017% Silwet L-77. Symptoms gradually appeared about two weeks after inoculation. Temperatures of 18-19° C. and high humidity (80%) was conducive for infection.

For X oryzae pv. oryzae inoculation, rice leaves were inoculated with the scissors clipping method, using cells suspended in sterile water at an OD600 of 0.5 containing erucamide or solvent. Lesion lengths were measured 12 days after inoculation.

### Purification and Identification of Erucamide

We performed the bioassay-guided purification of the active compounds by inhibiting AvrPto protein secretion of P. syringae DC3000 bacteria. Four-week-old A. thaliana leaves (107 g) were grinded into powder under liquid nitrogen, then extracted with 75% EtOH under sonication. The solvent was collected and removed under reduced pressure. The acquired crude extracted was suspended in water and partitioned successively with petroleum ether (PE), cyclohexane (CYH), methylene chloride (MC) and ethyl acetate (EA). The active MC fraction was applied to Sephodex LH-20 with MeOH/MC=1:10 to acquire six fractions, BA-E. The active subfraction C was purified by column chromatography on silica gel in dichloromethane/MeOH (100:1) to yield six sucfractions, C₁-C₆. The active C₃ group was further purified by column chromatography on silica gel in PE/EA (5:1) to give active fraction 9, which was characterized as erucamide. 1H NMR (500 MHz, Methanol-d4) δ 5.34 (ddd, J=5.6, 4.4, 1.1 Hz, 2H), 4.59 (s, 1H), 3.34 (s, 1H), 2.23-2.15 (m, 2H), 2.09-1.98 (m, 4H), 1.65-1.56 (m, 2H), 1.45-1.24 (m, 29H), 0.90 (t, J=7.0 Hz, 3H); 13C NMR (125 MHz, Methanol-d4) δ 178.20 129.46, 35.14, 29.43, 29.40-29.28 (m), 29.28-29.16 (m), 29.06, 28.93, 26.70, 25.52, 22.34, 13.04; HRMS (ESI): [M+H]+ calculated for C22H44NO: 338.3417, found: 338.3417.

### Quantification of Erucamide in Leaves

The quantification of erucamide in Arabidopsis leaves was performed with GC-TQ8050. The separation was achieved on a SH-Rxi-5Sil MS column (0.25 mm*30 m) in splitless mode at 290° C. The oven temperature program started at 180° C. for 3 min; then it was increased at a rate of 20° C. min-1 to 310° C. and held at 310° C. for 4 min. The carrier gas was helium at a flow rate of 2.0 ml min-1. The ion source was heated to 230° C.

Before injection, the sample was prepared by extracting 100 mg of Arabidopsis. Thaliana leaves with 2 ml of MC/MeOH (2:1) two times. To the extraction solvent 500 ng of d35 stearic acid amide was added as internal standard. The combined organic layer was removed under reduced pressure and re-dissolved with 1 ml of methylene chloride. After centrifugation to remove insoluble debris, the samples were ready for analysis.

### Analysis of TTSS Gene Expression and Type III Secretion

To determine AvrPto protein accumulation and secretion, Pst DC3000 was grown overnight in King's B medium at 28° C. Bacteria was harvested and washed twice with minimal medium [50 mM KH2PO4 (pH5.7), 7.6 mM (NH4)2SO4, 1.7 mM MgCl2.6H2O, 1.7 mM NaCl, 10 mM Fructose]. Then the bacteria were diluted to an OD600 of 0.4 in the minimal medium containing fructose and incubated in the presence of erucamide or solvent at 16° C. for 20 h. Total proteins from bacteria cell and culture supernatants were analyzed by immunoblot using anti-AvrPto antibodies as described. For control, anti-RNAP antibodies (Thermo Fisher Scientific) were used.

To determine AvrAC protein accumulation and secretion, Xcc8004 strain expressing HA-tagged AvrAC was cultivated at 28° C. in NYG rich medium (3 g/L yeast extract, 5 g/L peptone, 20 g/L glycerol, pH 7.0). Overnight cultured Xcc8004 were resuspended to an OD600 of 0.4 in minimal medium XCM2 [20 mM succinic acid, 0.15 g/L casamino acids, 7.57 mM (NH₄)₂SO₄, 0.01 mM MgSO₄, 60.34 mM K₂HPO₄, 33.07 mM KH₂PO₄, pH 6.6] and incubated in the presence of erucamide or solvent for 20 h. Total and supernatants protein were analyzed by immunoblot by indicated antibodies.

To determine SopF protein accumulation and secretion, Salmonella Typhimurium 14028S expressing His-tagged SopF was cultivated at 37° C. in LB medium and then subcultured (1:100) in fresh 30 mL LB medium with erucamide or solvent to an OD600 of 0.2. After induction with 0.5 mM IPTG, whole cells and culture supernatants were separated by centrifugation at 5000 RPM for 10 min. The pellet was resuspended by PBS as cytoplasmic protein sample. The supernatants were filtered (0.22 μm pore-size) and then precipitated by adding trichloroacetic acid at a final concentration of 10% v/v, followed by incubation at 4° C. overnight and centrifugation (20000 g, 4° C., 30 min). The pellet was washed twice by cold acetone and analyzed by immunoblot using anti-His antibodies. For control, anti-DnaK antibodies were used.

To determine the transcription of TTSS gene, P. syringae bacteria were treated as above. Total RNA was extracted using RNeasy Mini Kit (QIAGEN) and treated with RNase free DNase (Promega) to remove DNA. Subsequently, RT-qPCR was performed by using SYBR FAST One-Step qRT-PCR Kits (KAPA). Transcript level was normalized to that of 16S RNA.

### Effector Translocation

Arabidopsis plants were infiltrated with the Pst DC300 carrying the avrpto-cyaA gene fusion suspended at an OD600 of 0.1 in 5 mM MES containing 100 μM IPTG. Leaf discs were harvested 4-6 hpi for cAMP quantification. The samples were ground in liquid nitrogen and resuspended in 0.1 M HCl. cAMP levels were determined using the Direct cAMP ELISA kit (Enzo Life Sciences).

### Transmission Electron Microscopy

Pst DC3000 was grown overnight in King's B liquid medium at 28° C. to logarithmic phase. Strains were washed by minimal medium and cultivated on solid minimal medium containing fructose with erucamide or solvent at 21° C. for 3 days. The colonies cultured on solid medium were picked by pipet tips and then suspended in sterile water. Cell samples were attached onto copper grids after glow discharge and were rinsed with sterile water for 4 times. After that, the pili or flagella were negatively stained using 0.15% uranyl acetate. Images were taken with the JEM-1400(80 kV) electron microscope.

### Subcellular Cell Fraction

To isolate the membrane fraction, bacterial cells were harvested, washed with 10 mM Tris-HCl at pH 8.0, centrifuged and the supernatants were carefully discarded. The cell pellets were then resuspended in spheroplast buffer (20 mM Tris pH 8.0, 20% sucrose, 1 mM Na-EDTA), incubated for 10 min and centrifuged. The pellets were then resuspended in ice hypotonic solution (sterile water) and the sample were incubated for 10 min on ice. After centrifugation, the resulting pellets were resuspended in 10 mM Tris-HCl at pH 8.0 and sonicated. After centrifugation, the supernatants were carefully transferred to new tubes and centrifuged again for 40 min. The pellets were washed by sterile water and centrifuged for another 40 min. The pellets were resuspended in water as membrane proteins.

1-30. (canceled)

31. A compound, or a stereoisomer or tautomer thereof, or a salt of any of the foregoing, wherein the compound is selected from the group consisting of: 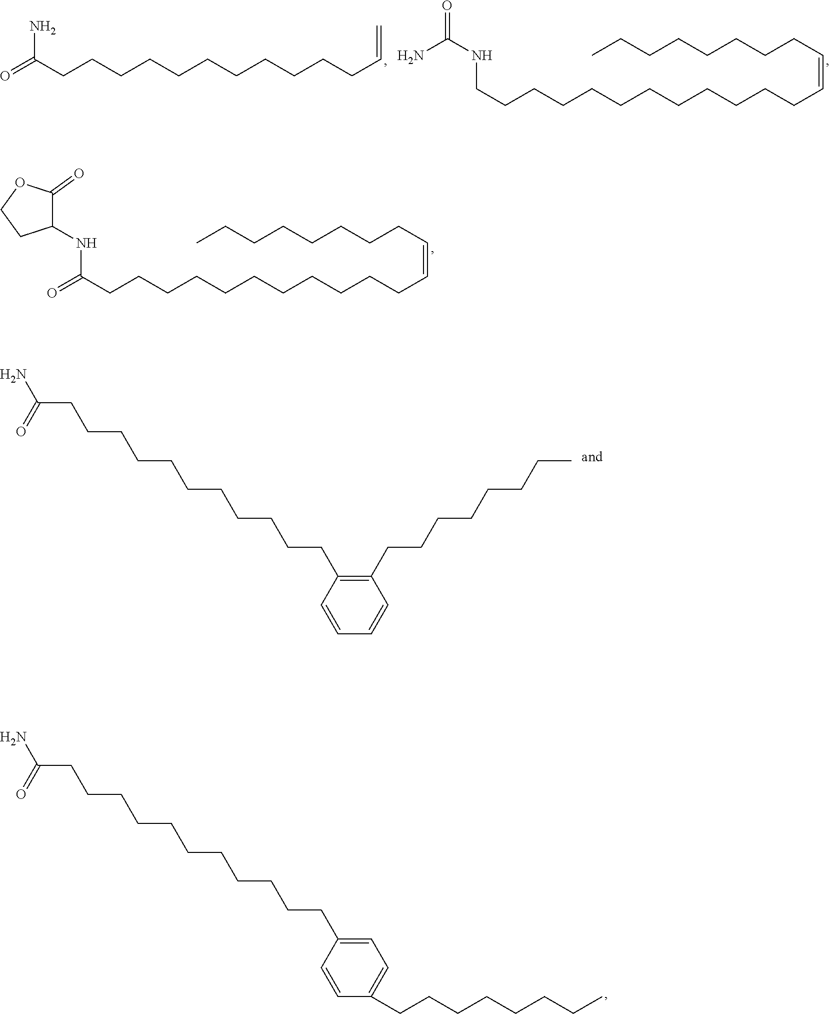
or a stereoisomer or tautomer thereof, or a salt of any of the foregoing.

32. A compound of formula (IA), or a stereoisomer or tautomer thereof, or a salt of any of the foregoing, 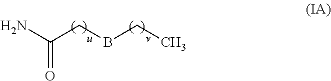
wherein: B is phenylene; u and v are each independently an integer of at least 1.

33. A composition comprising a compound of formula (IA) of claim 32, or formula (I), or a stereoisomer or tautomer thereof, or a salt of any of the foregoing, wherein the composition inhibits pathogenesis of gram-negative bacteria without killing the bacteria, 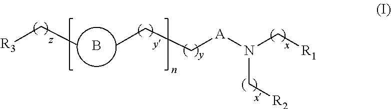
wherein:
x is an integer selected from 0-8;
x′ is an integer selected from 0-8;
each of R₁ and R₂ is independently —Rₐ, —ORₐ, —N(Rₐ)₂, —NHC(O)Rₐ, —NHS(O)₂Rₐ, —NHS(O)₂N(Rₐ)₂, —C(O)ORₐ, —OC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, nitro, cyano, or halogen, wherein each Rₐ is independently hydrogen, C₁₋₇ alkyl, C₃₋₈ cycloalkyl, 3-12 membered heterocyclyl, C₆₋₁₂ aryl, or 5-12 membered heteroaryl, amino; wherein the 3-12 membered heterocyclyl of Rₐ is optionally substituted with one or more oxo;
R₃ is independently —Rₐ, —ORₐ, —N(Rₐ)₂, —NHC(O)Rₐ, —NHS(O)₂Rₐ, —NHS(O)₂N(Rₐ)₂, —C(O)ORₐ, —OC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, nitro, cyano, or halogen, wherein each Rₐ is independently hydrogen, —CH═CH₂, —C≡CH, C₁₋₇ alkyl, C₃₋₈ cycloalkyl, 3-12 membered heterocyclyl, C₆₋₁₂ aryl, or 5-12 membered heteroaryl, amino;
A is a chemical bond, —C(O)—, —C(S)—, or —NHC(O)—;
y, y′, and z are each independently integer selected from 0-15;
B is independently —CH═CH—, —C≡C—, C₁₋₄ alkyl, C₃₋₈ cycloalkyl, 3-12 membered heterocyclyl, C₆₋₁₂ aryl, or 5-12 membered heteroaryl, each of which is independently optionally substituted by one or more R_{b}, wherein each R_{b} is independently C₁₋₆ alkyl, C₁₋₆ alkoxyl, C₃₋₈ cycloalkyl, 3-12 membered heterocyclyl, C₆₋₁₂ aryl, 5-12 membered heteroaryl, amino, hydroxyl, carboxyl, nitro, cyano, or halogen;
n is an integer selected from 0-8.

34. The composition of claim 33, wherein the compound is selected from the group consisting of: 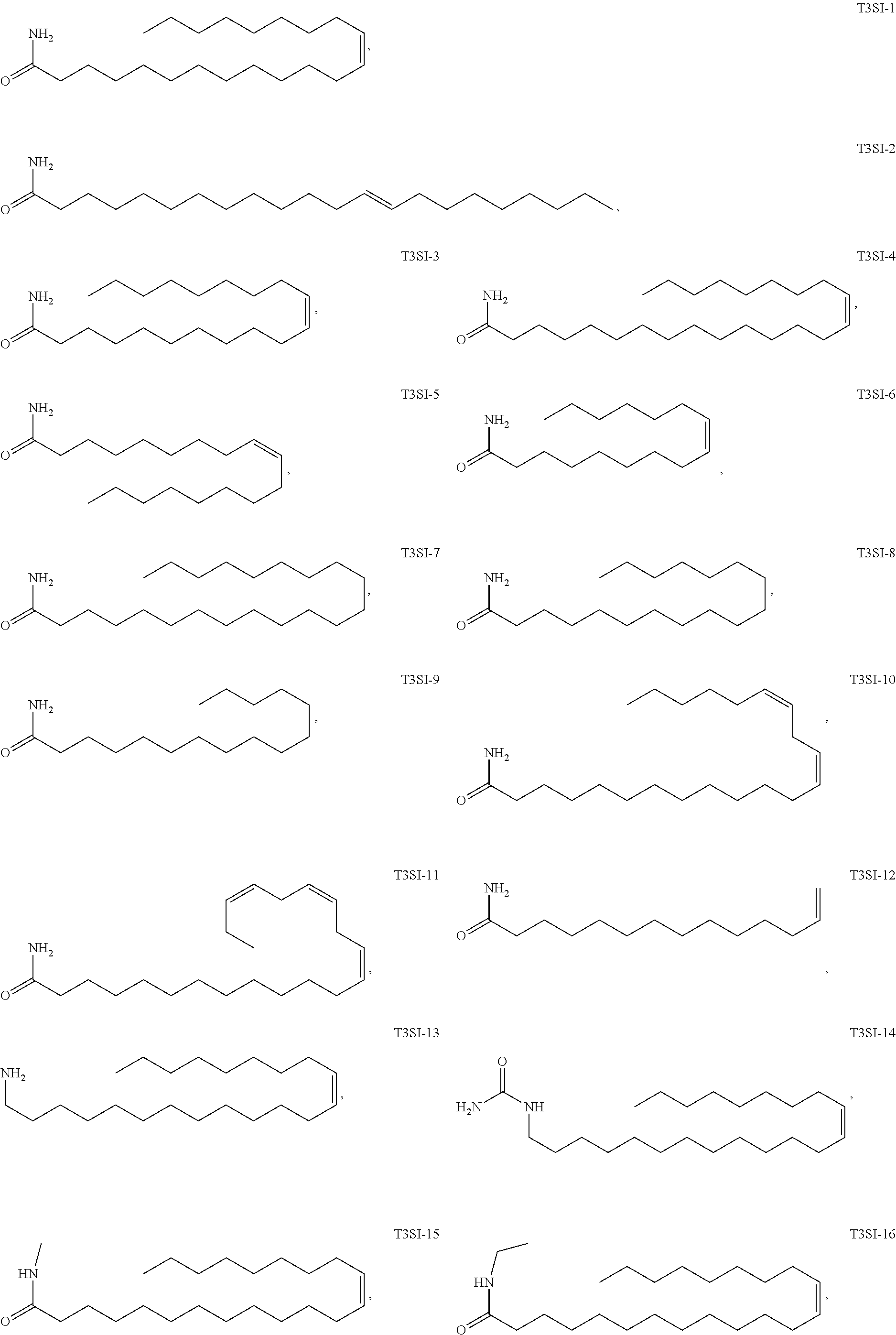 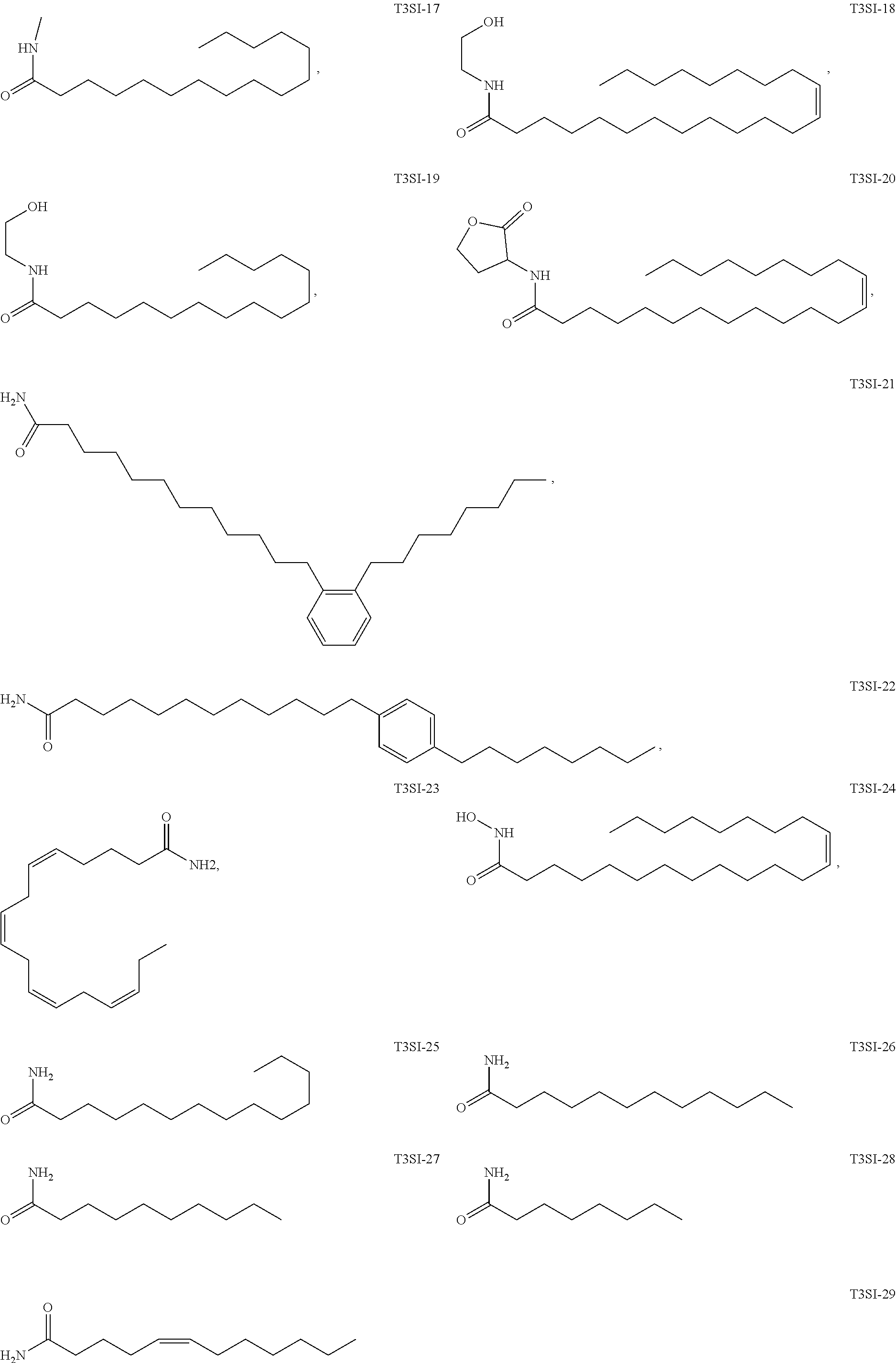
or a stereoisomer or tautomer thereof, or a salt of any of the foregoing.

35. The composition of claim 33, wherein composition comprises at least about 20% (w/w) of the compound; and/or
the composition comprises no more than about 1000 (w/w) of the compound; and/or
the composition comprises no more than about 60 μmol of the compound; and/or
the composition is substantially free of bacteriocides; and/or
the composition inhibits secretion of Type III secretion system (TTSS) of the gram-negative bacteria; and/or
the composition is substantially free of a chemically synthesized ingredient; and/or
wherein the compound is erucamide.

36. A method for preventing and/or treating infection of a host species by bacterial pathogens, wherein said method comprises administering an effective amount of a composition of claim 33 to the host species.

37. The method of claim 36, wherein the host species is a plant species.

38. The method of claim 37, wherein the plant is selected from the group consisting of a solanaceous plant, a leguminous plant, a cruciferous plant, a gramineous plant, a cucurbitaceous plant, a liliaceous plant, and a rutaceous plant, a poaceae plant, an araliaceae plant.

39. The method of claim 38, wherein the plant is a solanaceous plant, ad wherein the solanaceous plant(s) is tomato, eggplant, potato, tobacco, bell pepper, or chili pepper.

40. The method of claim 37, wherein the plant is selected from the group consisting of a fruit tree, a horticultural tree and an ornamental plant.

41. The method of claim 37, wherein the composition is administered to the plant by foliar administration, spraying, stem-coating, clipping, emersion, or watering; or
the composition is administered by coating the seeds of the plant with the composition prior to sowing the seeds; or
the composition is administered to the plant by adding the composition to the medium in which the plant is growing.

42. The method of claim 36, wherein the host species is an animal species.

43. The method of claim 42, wherein the animal species is a domesticated or agricultural animal.

44. The method of claim 42, wherein the animal species is an insect species of agricultural importance, for example the honey bee.

45. The method of claim 42, wherein the animal species is an aquatic animal or a poultry animal.

46. The method of claim 42, wherein the composition is administered orally, by infusion, injection, or inhalation.

47. The method of claim 36, wherein said Gram-negative bacterium is of the genus Pseudomonas, Xanthomonas, Ralstonia, Salmonella, Shigella, Escherichia, Burkholderia, Yersinia, Erwinia, Dickeya or Chlamydia.

48. The method according to claim 36, wherein said gram-negative bacteria are Pseudomonas syringae, Pseudomonas aeruginosa, Pseudomonas amygdali, Pseudomonas avellanae, Pseudomonas caricapapayae, Pseudomonas cichorii, Pseudomonas coronafaciens, Pseudomonas ficuserectae, Pseudomonas helianthi, Pseudomonas meliae, Pseudomonas savastanoi, Pseudomonas tomato, Pseudomonas viridiflava, Pseudomonas asplenii, Pseudomonas cannabina, Pseudomonas costantinii, Pseudomonas fuscovaginae, Pseudomonas suis, Pseudomonas marginalis, Pseudomonas mediterranea, Xanthomonas albilineans, Xanthomonas alfalfae, Xanthomonas ampelina, Xanthomonas arboricola, Xanthomonas axonopodis, Xanthomonas boreopolis, Xanthomonas badrii, Xanthomonas bromi, Xanthomonas campestris, Xanthomonas cassavae, Xanthomonas citri, Xanthomonas codiaei, Xanthomonas cucurbitae, Xanthomonas cyanopsidis, Xanthomonas cynarae, Xanthomonas euvesicatoria, Xanthomonas fragariae, Xanthomonas gardneri, Xanthomonas holcicola, Xanthomonas hortorum, Xanthomonas hyacinthi, Xanthomonas maliensis, Xanthomonas malvacearum, Xanthomonas maltophila, Xanthomonas manihotis, Xanthomonas melonis, Xanthomonas oryzae, Xanthomonas papavericola, Xanthomonas perforans, Xanthomonas phaseoli, Xanthomonas pisi, Xanthomonas populi, Xanthomonas sacchari, Xanthomonas theicola, Xanthomonas translucens, Xanthomonas vasicola, Xanthomonas vesicatoria, Ralstonia solanacearum, Salmonella enterica, Shigella dysenteriae, Enterohaemorrhagic Escherichia coli, Burkholderia pseudomallei, Burkholderia dolosa, Burkholderia gladioli, Burkholderia glumae, Burkholderia mallei, Burkholderia multivorans, Burkholderia oklahomensis, Burkholderia pseudomallei, Burkholderia vietnamiensis, Yersinia pestis, Erwinia amylovora, Dickeya dadanthii, Erwinia carotovora, Erwinia papayae, Erwinia psidii, Erwinia pyrifoliae, Erwinia tracheiphila, Dickeya solani or Chlamydia trachomatis.

49. The method according to claim 36, wherein said gram-negative bacteria are Pseudomonas syringae pv. maculicola, Pseudomonas syringae pv. tomato, Pseudomonas syringae pv. glycinea, Pseudomonas syringae pv. lachrymans, Pseudomonas syringae pv. apii, Pseudomonas syringae pv. phaseolicola, Pseudomonas syringae pv. aceris, Pseudomonas syringae pv. actinidiae, Pseudomonas syringae pv. aesculi, Pseudomonas syringae pv. aptata, Pseudomonas syringae pv. coronafaciens, Pseudomonas syringae pv. japonica, Pseudomonas syringae pv. morsprunorum, Pseudomonas syringae pv. tabaci, Pseudomonas syringae pv. syringae, Pseudomonas amygdai pv. aesculi, Pseudomonas amygdai pv. amygdai, Pseudomonas amygdai pv. ciccaronei, Pseudomonas amygdai pv. dendropanacis, Pseudomonas amygdai pv. eriobotryae, Pseudomonas amygdai pv. glycinea, Pseudomonas amygdahi pv. hibisci, Pseudomonas amygdai pv. lachrymans, Pseudomonas amygdai pv. mellea, Pseudomonas amygdali pv. mori, Pseudomonas amygdali pv. morsprumorum, Pseudomonas amygdali pv. myricae, Pseudomonas amygdali pv. phaseolicola, Pseudomonas amygdali pv. photiniae, Pseudomonas amygdali pv. sesami, Pseudomonas amygdali pv. tabaci, Pseudomonas amygdali pv. ulmi, Pseudomonas coronafaciens pv. atropurpurea, Pseudomonas coronafaciens pv. coronafaciens, Pseudomonas coronafaciens pv. garcae, Pseudomonas coronafaciens pv. oryzae, Pseudomonas coronafaciens pv. porri, Pseudomonas coronafaciens pv. striafaciens, Pseudomonas coronafaciens pv. zizaniae, Pseudomonas helianthi pv. helianthi, Pseudomonas helianthi pv. tagetis, Pseudomonas savastanoi pv. fraxini, Pseudomonas savastanoi pv. nerii, Pseudomonas savastanoi. pv. oleae, Pseudomonas savastanoi pv. phaseolicola, Pseudomonas savastanoi pv. savastanoi, Pseudomonas tomato pv. antirrhini, Pseudomonas tomato pv. apii, Pseudomonas tomato pv. berberidis, Pseudomonas tomato pv. delphinii, Pseudomonas tomato pv. lachrymans, Pseudomonas tomato pv. maculicola, Pseudomonas tomato pv. morsprunorum, Pseudomonas tomato pv. passiflorae, Pseudomonas tomato pv. persicae, Pseudomonas tomato pv. philadelphi, Pseudomonas tomato pv. primulae, Pseudomonas tomato pv. ribicola, Pseudomonas tomato pv. tomato, Pseudomonas tomato pv. viburni, Pseudomonas viridiflava pv. primulae, Pseudomonas viridiflava pv. ribicola, Pseudomonas viridiflava pv. viridiflava, Pseudomonas marginalis pv. alfalfa, Pseudomonas marginalis pv. marginalis, Pseudomonas marginalis. pv. pastinacae, Xanthomonas campestris pv. armoraciae, Xanthomonas campestris pv. begonia, Xanthomonas campestris pv. begoniae, Xanthomonas campestris pv. campestris, Xanthomonas campestris pv. cannabis, Xanthomonas campestris pv. carota, Xanthomonas campestris pv. corylina, Xanthomonas campestris pv. dieffenbachiae, Xanthomonas campestris pv. glycines, Xanthomonas campestris pv. graminis, Xanthomonas campestris pv. hederae, Xanthomonas campestris pv. hyacinthi, Xanthomonas campestris pv. juglandis, Xanthomonas campestris pv. malvacearum, Xanthomonas campestris pv. musacearum, Xanthomonas campestris pv. mangiferaeindicae, Xanthomonas campestris pv. mori, Xanthomonas campestris pv. nigromaculans, Xanthomonas campestris pv. pelargonii, Xanthomonas campestris pv. phaseoli, Xanthomonas campestris pv. poinsettiicola, Xanthomonas campestris pv. pruni, Xanthomonas campestris pv. raphanin, Xanthomonas campestris pv. sesame, Xanthomonas campestris pv. tardicrescens, Xanthomonas campestris pv. translucens, Xanthomonas campestris pv. vesicatoria, Xanthomonas campestris pv. viticola, Xanthomonas oryzae pv. oryzae, Xanthomonas oryzae pv. oryzicola, S Salmonella Choleraesuis, Salmonella Dublin, Salmonella Enteritidis, Salmonella Gallinarum, Salmonella Hadar, Salmonella Heidelberg, Salmonella Infantis, Salmonella Paratyphi, Salmonella typhi, Salmonella Typhimurium, enterotoxigenic Escherichia coli, enteropathogenic Escherichia coli, enteroinvasive Escherichia coli, enteroaggregative Escherichia coli, Burkholderia gladioli pv. gladioli, Burkholderia gladioli pv. allicola or Burkholderia gladioli pv. agaricicola.

50. A method of preparing the compound of claim 31, or a stereoisomer or tautomer thereof, or a salt of any of the foregoing.
